SEQUENCE LISTING (21 sequences)

SEQ ID NO: 1 - DNA, 450 bp, Human Adenovirus Ad5
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatag

SEQ ID NO: 2 - DNA, 2282 bp, Human Adenovirus Ad5
gtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcga

SEQ ID NO: 3 - DNA, 1585 bp, Human Adenovirus Ad5
aaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggc

SEQ ID NO: 4 - DNA, 610 bp, Human Adenovirus Ad5
agcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttca

SEQ ID NO: 5 - DNA, 548 bp, Human Adenovirus Ad5
gatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcgg

SEQ ID NO: 6 - DNA, 1487 bp, Human Adenovirus Ad5
gcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacggga

SEQ ID NO: 7 - DNA, 549 bp, Human Adenovirus Ad5
actcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataa

SEQ ID NO: 8 - DNA, 885 bp, Human Adenovirus Ad5
ctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcat

SEQ ID NO: 9 - DNA, 30 bp, Artificial Sequence
  Synthetic Oligonucleotide
ttatacgcgtgccaccatgactacgtccgg

SEQ ID NO: 10 - DNA, 28 bp, Artificial Sequence
  Synthetic Oligonucleotide
ttatgctagcgcgaaggagaagtccacg

SEQ ID NO: 11 - DNA, 1590 bp, Human Adenovirus Ad5
ttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccat

SEQ ID NO: 12 - DNA, 1980 bp, Human Adenovirus Ad5
gcatgcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcattggtgtaagagcttcagccaagagttacacataaagg

SEQ ID NO: 13 - DNA, 22 bp, Artificial Sequence
  Synthetic Oligonucleotide
aactacaattcccaacacatac

SEQ ID NO: 14 - DNA, 19 bp, Artificial Sequence
  Synthetic Oligonucleotide
cacatccgtcgcttacatg

SEQ ID NO: 15 - DNA, 34 bp, Artificial Sequence
  Synthetic
gcctagggatgcgtgtcataatcagtgtgggttc

SEQ ID NO: 16 - DNA, 38 bp, Artificial Sequence
  Synthetic
caattgaaaagtgagcgggaagagctggaagaaccatg

SEQ ID NO: 17 - DNA, 9033 bp, Human Hepatitis C Virus
atgagcacgaatcctaaacctcaaagaaaaaccaaacgtaacaccaaccgccgcccacaggacgtcaagttcccgggcggtggtcagatcgttggtggagtttacctgttgccgcgcaggggccccaggttgggtgtgcgcgcgactaggaagacttccgagcggtcgcaacctcgtggaaggcgacaacctatccccaaggctcgccagcccgagggcagggcctgggctcagcccgggtacccttggcccctctatggcaatgagggcatggggtgggcaggatggctcctgtcaccccgcggctctcggcctagttggggccccacggacccccggcgtaggtcgcgtaatttgggtaaggtcatcgataccctcacatgcggcttcgccgatctcatggggtacattccgctcgtcggcgcccccctggggggcgctgccagggccctggcacatggtgtccgggttctggaggacggcgtgaactatgcaacagggaatctgcccggttgctctttttctatcttcctcttggctctgctgtcctgcctgaccaccccagcttccgcttacgaagtgcacaacgtgtccgggatatatcatgtcacgaacgactgctccaacgcaagcattgtgtatgaggcagcggacttgatcatgcatactcctgggtgcgtgccctgcgttcgggaaggcaactcctcccgctgctgggtagcgctcactcccacgctcgcagccaggaacgtcaccatccccaccacgacgatacgacgccacgtcgatctgctcgttggggcggctgctttctgttccgctatgtacgtgggggacctctgcggatctgttttcctcgtctctcagctgttcaccttctcgcctcgccggcatgtgacattacaggactgtaactgctcaatttatcccggccatgtgtcgggtcaccgtatggcttgggacatgatgatgaactggtcgcccacaacagccctagtggtgtcgcagttactccggatcccacaagccgtcgtggacatggtggcgggggcccactggggagtcctggcgggccttgcctactattccatggcggggaactgggctaaggttctgattgtgatgctactttttgctggcgttgacggggatacccacgtgacagggggggcgcaagccaaaaccaccaacaggctcgtgtccatgttcgcaagtgggccgtctcagaaaatccagcttataaacaccaatggcagttggcacatcaacaggactgccctgaactgcaatgactctctccagactgggtttcttgccgcgctgttctacacacatagtttcaactcgtccgggtgcccagagcgcatggccagctgccgcaccattgacaagttcgaccagggatggggtcccattacttatgctgagtctagcagatcagaccagaggccatattgctggcactacccacctccacaatgtaccatcgtacctgcgtcggaggtgtgcggcccagtgtactgcttcaccccaagccctgtcgtcgtggggacgaccgatcgtttcggtgtccctacgtatagatggggggagaacgagactgacgtgctgctgctcaacaacacgcggccgccgcaaggcaactggttcggctgcacatggatgaatagcaccgggttcaccaagacatgtggggggcccccgtgtaacatcgggggggtcggcaacaacaccctgacctgccccacggactgcttccggaagcaccccgaggctacctacacaaaatgtggttcggggccttggctgacacctaggtgcatggttgactatccatacaggctctggcattacccctgcactgttaactttaccatcttcaaggttaggatgtatgtggggggcgtggagcacaggctcaatgctgcatgcaattggacccgaggagagcgttgtgacttggaggacagggatagggcggagctcagcccgctgctgctgtctacaacagagtggcaggtactgccctgttccttcaccaccctaccagctctgtccactggcttgattcacctccatcagaacatcgtggacgtgcaatacctatacggtatagggtcagcggttgtctcctttgcaatcaaatgggagtatgtcctgttgcttttccttctcctagcggacgcacgtgtctgtgcctgcttgtggatgatgctgctgatagcccaggccgaggccgccttggagaacctggtggtcctcaatgcggcgtctgtggccggcgcacatggcatcctctccttccttgtgttcttctgtgccgcctggtacatcaaaggcaggctggtccctggggcggcatatgctctttatggcgtgtggccgctgctcctgctcttgctggcattaccaccgcgagcttacgccatggaccgggagatggctgcatcgtgcggaggcgcggtttttgtgggtctggtactcctgactttgtcaccatactacaaggtgttcctcgctaggctcatatggtggttacaatattttaccaccagagccgaggcgcacttacatgtgtggatcccccccctcaacgctcggggaggccgcgatgccatcatcctcctcatgtgcgcagtccatccagagctaatctttgacatcaccaaacttctaattgccatactcggtccgctcatggtgctccaagctggcataaccagagtgccgtacttcgtgcgcgctcaagggctcattcatgcatgcatgttagtgcggaaggtcgctgggggtcattatgtccaaatggccttcatgaagctgggcgcgctgacaggcacgtacatttacaaccatcttaccccgctacgggattgggcccacgcgggcctacgagaccttgcggtggcagtggagcccgtcgtcttctccgacatggagaccaagatcatcacctggggagcagacaccgcggcgtgtggggacatcatcttgggtctgcccgtctccgcccgaaggggaaaggagatactcctgggcccggccgatagtcttgaagggcgggggtggcgactcctcgcgcccatcacggcctactcccaacagacgcggggcctacttggttgcatcatcactagccttacaggccgggacaagaaccaggtcgagggagaggttcaggtggtttccaccgcaacacaatccttcctggcgacctgcgtcaacggcgtgtgttggaccgtttaccatggtgctggctcaaagaccttagccggcccaaaggggccaatcacccagatgtacactaatgtggaccaggacctcgtcggctggcaggcgccccccggggcgcgttccttgacaccatgcacctgtggcagctcagacctttacttggtcacgagacatgctgacgtcattccggtgcgccggcggggcgacagtagggggagcctgctctcccccaggcctgtctcctacttgaagggctcttcgggtggtccactgctctgcccttcggggcacgctgtgggcatcttccgggctgccgtatgcacccggggggttgcgaaggcggtggactttgtgcccgtagagtccatggaaactactatgcggtctccggtcttcacggacaactcatcccccccggccgtaccgcagtcatttcaagtggcccacctacacgctcccactggcagcggcaagagtactaaagtgccggctgcatatgcagcccaagggtacaaggtgctcgtcctcaatccgtccgttgccgctaccttagggtttggggcgtatatgtctaaggcacacggtattgaccccaacatcagaactggggtaaggaccattaccacaggcgcccccgtcacatactctacctatggcaagtttcttgccgatggtggttgctctgggggcgcttatgacatcataatatgtgatgagtgccattcaactgactcgactacaatcttgggcatcggcacagtcctggaccaagcggagacggctggagcgcggcttgtcgtgctcgccaccgctacgcctccgggatcggtcaccgtgccacacccaaacatcgaggaggtggccctgtctaatactggagagatccccttctatggcaaagccatccccattgaagccatcagggggggaaggcatctcattttctgtcattccaagaagaagtgcgacgagctcgccgcaaagctgtcaggcctcggaatcaacgctgtggcgtattaccgggggctcgatgtgtccgtcataccaactatcggagacgtcgttgtcgtggcaacagacgctctgatgacgggctatacgggcgactttgactcagtgatcgactgtaacacatgtgtcacccagacagtcgacttcagcttggatcccaccttcaccattgagacgacgaccgtgcctcaagacgcagtgtcgcgctcgcagcggcggggtaggactggcaggggtaggagaggcatctacaggtttgtgactccgggagaacggccctcgggcatgttcgattcctcggtcctgtgtgagtgctatgacgcgggctgtgcttggtacgagctcacccccgccgagacctcggttaggttgcgggcctacctgaacacaccagggttgcccgtttgccaggaccacctggagttctgggagagtgtcttcacaggcctcacccacatagatgcacacttcttgtcccagaccaagcaggcaggagacaacttcccctacctggtagcataccaagccacggtgtgcgccagggctcaggccccacctccatcatgggatcaaatgtggaagtgtctcatacggctgaaacctacgctgcacgggccaacacccttgctgtacaggctgggagccgtccaaaatgaggtcaccctcacccaccccataaccaaatacatcatggcatgcatgtcggctgacctggaggtcgtcactagcacctgggtgctggtgggcggagtccttgcagctctggccgcgtattgcctgacaacaggcagtgtggtcattgtgggtaggattatcttgtccgggaggccggctattgttcccgacagggagtttctctaccaggagttcgatgaaatggaagagtgcgcctcgcacctcccttacatcgagcagggaatgcagctcgccgagcaattcaagcagaaagcgctcgggttactgcaaacagccaccaaacaagcggaggctgctgctcccgtggtggagtccaagtggcgagcccttgagacattctgggcgaagcacatgtggaatttcatcagcgggatacagtacttagcaggcttatccactctgcctgggaaccccgcaatagcatcattgatggcattcacagcctctatcaccagcccgctcaccacccaaagtaccctcctgtttaacatcttgggggggtgggtggctgcccaactcgccccccccagcgccgcttcggctttcgtgggcgccggcatcgccggtgcggctgttggcagcataggccttgggaaggtgcttgtggacattctggcgggttatggagcaggagtggccggcgcgctcgtggccttcaaggtcatgagcggcgagatgccctccaccgaggacctggtcaatctacttcctgccatcctctctcctggcgccctggtcgtcggggtcgtgtgtgcagcaatactgcgtcgacacgtgggtccgggagagggggctgtgcagtggatgaaccggctgatagcgttcgcctcgcggggtaatcatgtttcccccacgcactatgtgcctgagagcgacgccgcagcgcgtgttactcagatcctctccagccttaccatcactcagctgctgaaaaggctccaccagtggattaatgaagactgctccacaccgtgttccggctcgtggctaagggatgtttgggactggatatgcacggtgttgactgacttcaagacctggctccagtccaagctcctgccgcagctaccgggagtcccttttttctcgtgccaacgcgggtacaagggagtctggcggggagacggcatcatgcaaaccacctgcccatgtggagcacagatcaccggacatgtcaaaaacggttccatgaggatcgtcgggcctaagacctgcagcaacacgtggcatggaacattccccatcaacgcatacaccacgggcccctgcacaccctctccagcgccaaactattctagggcgctgtggcgggtggccgctgaggagtacgtggaggtcacgcgggtgggggatttccactacgtgacgggcatgaccactgacaacgtaaagtgcccatgccaggttccggctcctgaattcttcacggaggtggacggagtgcggttgcacaggtacgctccggcgtgcaggcctctcctacgggaggaggttacattccaggtcgggctcaaccaatacctggttgggtcacagctaccatgcgagcccgaaccggatgtagcagtgctcacttccatgctcaccgacccctcccacatcacagcagaaacggctaagcgtaggttggccagggggtctcccccctccttggccagctcttcagctagccagttgtctgcgccttccttgaaggcgacatgcactacccaccatgtctctccggacgctgacctcatcgaggccaacctcctgtggcggcaggagatgggcgggaacatcacccgcgtggagtcggagaacaaggtggtagtcctggactctttcgacccgcttcgagcggaggaggatgagagggaagtatccgttccggcggagatcctgcggaaatccaagaagttccccgcagcgatgcccatctgggcgcgcccggattacaaccctccactgttagagtcctggaaggacccggactacgtccctccggtggtgcacgggtgcccgttgccacctatcaaggcccctccaataccacctccacggagaaagaggacggttgtcctaacagagtcctccgtgtcttctgccttagcggagctcgctactaagaccttcggcagctccgaatcatcggccgtcgacagcggcacggcgaccgcccttcctgaccaggcctccgacgacggtgacaaaggatccgacgttgagtcgtactcctccatgcccccccttgagggggaaccgggggaccccgatctcagtgacgggtcttggtctaccgtgagcgaggaagctagtgaggatgtcgtctgctgctcaatgtcctacacatggacaggcgccttgatcacgccatgcgctgcggaggaaagcaagctgcccatcaacgcgttgagcaactctttgctgcgccaccataacatggtttatgccacaacatctcgcagcgcaggcctgcggcagaagaaggtcacctttgacagactgcaagtcctggacgaccactaccgggacgtgctcaaggagatgaaggcgaaggcgtccacagttaaggctaaactcctatccgtagaggaagcctgcaagctgacgcccccacattcggccaaatccaagtttggctatggggcaaaggacgtccggaacctatccagcaaggccgttaaccacatccactccgtgtggaaggacttgctggaagacactgtgacaccaattgacaccaccatcatggcaaaaaatgaggttttctgtgtccaaccagagaaaggaggccgtaagccagcccgccttatcgtattcccagatctgggagtccgtgtatgcgagaagatggccctctatgatgtggtctccacccttcctcaggtcgtgatgggctcctcatacggattccagtactctcctgggcagcgagtcgagttcctggtgaatacctggaaatcaaagaaaaaccccatgggcttttcatatgacactcgctgtttcgactcaacggtcaccgagaacgacatccgtgttgaggagtcaatttaccaatgttgtgacttggcccccgaagccagacaggccataaaatcgctcacagagcggctttatatcgggggtcctctgactaattcaaaagggcagaactgcggttatcgccggtgccgcgcgagcggcgtgctgacgactagctgcggtaacaccctcacatgttacttgaaggcctctgcagcctgtcgagctgcgaagctccaggactgcacgatgctcgtgaacgccgccggccttgtcgttatctgtgaaagcgcgggaacccaagaggacgcggcgagcctacgagtcttcacggaggctatgactaggtactctgccccccccggggacccgccccaaccagaatacgacttggagctgataacatcatgttcctccaatgtgtcggtcgcccacgatgcatcaggcaaaagggtgtactacctcacccgtgatcccaccacccccctcgcacgggctgcgtgggaaacagctagacacactccagttaactcctggctaggcaacattatcatgtatgcgcccactttgtgggcaaggatgattctgatgactcacttcttctccatccttctagcacaggagcaacttgaaaaagccctggactgccagatctacggggcctgttactccattgagccacttgacctacctcagatcattgaacgactccatggccttagcgcattttcactccatagttactctccaggtgagatcaatagggtggcttcatgcctcaggaaacttggggtaccacccttgcgagtctggagacatcgggccaggagcgtccgcgctaggctactgtcccagggggggagggccgccacttgtggcaagtacctcttcaactgggcagtgaagaccaaactcaaactcactccaatcccggctgcgtcccagctggacttgtccggctggttcgttgctggttacagcgggggagacatatatcacagcctgtctcgtgcccgaccccgctggttcatgctgtgcctactcctactttctgtaggggtaggcatctacctgctccccaaccgataa

SEQ ID NO: 18 - DNA, 28 bp, Artificial Sequence
  Synthetic Probe
tggcatgacactacgaccaacacgatct

SEQ ID NO: 19 - DNA, 19 bp, Artificial Sequence
  Synthetic Primer
actacgtccggcgttccat

SEQ ID NO: 20 - DNA, 18 bp, Artificial Sequence
  Synthetic Primer
ggagtgcgccgagacaac

SEQ ID NO: 21 - protein, 9 aa, Human Hepatitis C Virus
GAVQNEVTL

(19) United States
(12) Patent Application Publication
(10) Pub. No.: US 2007/0298498 A1
(43) Pub. Date: Dec. 27, 2007

(54) Adenoviral Amplicon and Producer Cells for the Production of Replication-Defective Adenoviral Vectors, Methods of Preparation and Use Thereof

(76) Inventors: Stefano Colloca, Rome (IT); Daniele Catalucci, San Diego, CA (US)

Correspondence Address: MERCK AND CO., INC, P O BOX 2000, RAHWAY, NJ, 07065-0907, US

(21) Appl. No.: 11/666,783

(22) PCT Filed: Oct. 27, 2005
(86) PCT No.: PCT/EP05/11629

§ 371 Date: May 1, 2007

Related U.S. Application Data
(60) Provisional application No. 60/624,459, filed on Nov. 2, 2004.

(51) Int. Cl. C12N 15/86; C07H 21/04; C12N 1/00; C12N 5/16; C12N 15/74

(52) U.S. Cl. 435/455; 435/243; 435/363; 435/477; 536/23.2

(57) ABSTRACT

The present invention relates to a plasmid that can be used for the development of efficient producer cell lines for the production of helper independent adenovirus vectors carrying multiple deletions of non-structural as well as structural genes. More specifically, the present invention provides producer cells which comprise a novel adenoviral amplicon that can be used to complement a multi-deleted adenoviral vectors and obtain high titer preparations. The amplicon is an episomal plasmid that expresses Ad5 E2 viral genes (i.e., polymerase, pre-terminal protein and DNA binding protein) and E4 orf6, the EBV the latent origin of replication (OriP) as well as adenoviral origins of replications in form of a covalent junction of left and right ITRs. This plasmid is capable of self-replication upon induction of Ad5 E2 gene expression. The invention further includes methods for the preparation of the disclosed producer cells and uses of the cells to produce viral vectors on a scale that is sufficient for therapeutic uses.

### CROSS-REFERENCE TO RELATED APPLICATIONS

This application claims the benefit of U.S. Provisional Application No. 60/624,459, filed Nov. 2, 2004, herein incorporated by reference.

### FIELD OF THE INVENTION

The present invention relates to the field of molecular biology and in particular to the development and use of an episomal plasmid, capable of inducible self-replication, to prepare high cloning capacity producer cell lines for the production of multi- or fully-deleted helper-independent adenoviral vectors.

### BACKGROUND OF THE INVENTION

Adenoviruses (Ads) are characterized by a broad tropism in that they are able to infect both quiescent and proliferating cells of a wide variety of tissues. Generally speaking, infection of a permissive cell with wild type human Ad5 virus results in the production of approximately 10⁴-10⁵ viral particles. The capacity for high titer propagation, together with ease of manipulation of the viral genome, makes Ad vectors attractive for use as gene transfer vectors for vaccination and gene therapy as well as for gene expression in cell culture.

Several vector systems based on human Ad5 and Ad2 have been developed with the goal of improving the safety profile (e.g., minimizing toxicity resulting from viral gene expression) and increasing the cloning capacity of the preceding generation of vectors. Strategies for developing alternative vector systems typically involve deleting adenoviral genes from the vector backbone. The adenoviral genome is functionally subdivided into early and late regions, comprising genes encoding non-structural and structural products. The first region comprises the Early (E) genes which encodes polypeptides expressed prior to viral DNA replication. The second region comprises the Late (L) genes which encode polypeptides required in the subsequent stages of viral replication. The L region of the adenoviral genome essentially encodes structural proteins required for the assembly of viral particles.

Following infection of a competent cell, the first region to be transcribed is the E1a region which codes for proteins involved in the transactivation of both E and L genes. The subsequently transcribed E1b region encodes polypeptides which regulate RNA synthesis, and protect the host cell from an apoptotic effect exerted by E1a. Therefore, the E1a/E1b genes/functions are essential for viral replication. First generation (FG) adenoviral vectors, typically include deletions in adenoviral E1 genes. These deletions render the adenovirus replication-defective, unless the protein products of the modified transcriptional units are provided in trans. Generally speaking, the maximum capacity of a FG adenoviral vector does not exceed 8 kb. Although, FG Ad5 vectors are attenuated by deleting or modifying the E1 region, cytotoxicity is commonly observed in vitro as a consequence of both leaky gene expression and retained capacity for replication in some tumor cell lines. Typically, in vivo transduction with a FG Ad vector produces a relatively short term transgene expression.

Second and third generation vector system, based on the deletion of additional viral genes resulted in further attenuation of adenoviral gene expression and increased vector capacity. More specifically, newer generation vectors comprise additional deletions in viral E2, E3 and/or E4 genes. The cloning capacity of a ΔE1/E3/E4 vector approaches about 11 kb. The E2 region encodes proteins that are directly involved in viral replication, including the viral DNA-polymerase, the pre-terminal protein and proteins binding to the viral DNA. The E3 region is known to encode proteins that are not required for viral replication, but which function in vivo to control the host immune response. The E4 region genes encode polypeptides that reduce the gene expression of the host cell and also function to increase the transcription of E2 and L region of the adenoviral genome. The use of multi-deleted vectors with E1, E2a/b, E3 and/or E4 deletions in different combinations have been observed to be less cytotoxic in vitro and more stable in mouse liver than classic FG (2-4,23,24,33,45,52) vectors. However, there is no conclusive evidence that the newer generation adenovirus vectors are capable of significantly prolonged persistence. Moreover, the introduction of additional deletions has significantly decreased the resulting titers, making the vectors more difficult to produce in large scale for clinical applications (33,18). In fact in nearly every case, the expression of complementing genes that are stably introduced into packaging/producer cell lines, is inefficient when multiple deletions must be complemented (5,54).

To date, helper dependent (HD) fully-deleted adenoviral vectors genes are considered to be one of the most efficient and safe vectors for in vivo gene transfer (5, 15, 28, 36, 3941, 43, 54). Fully-deleted Ad vectors contain only the cis elements necessary for replication and packaging (i.e., encapsidation), but lack all adenoviral genes. Traditionally, the requisite adenoviral genes are provided in trans by a helper virus. However, HD vectors are characterized by a number of disadvantages. Among these is the requirement for control of three independent components because the system requires a co-infection of a packaging cell line with a HD vector carrying a transgene and a helper virus that provides the necessary virus proteins in trans. In practice, production of a helper-dependent adenoviral vector on a pharmaceutical scale entails difficulties that are hard to overcome and production costs that are too high. In addition, the use a helper virus almost always contaminates HD vectors preparations.

Multi-deleted helper independent, Ad vectors have been also been constructed by deleting some of the E2 genes and/or the E4 region, or combining deletions of different early genes (2-4, 23, 24, 33, 45, 52). Typically, the requisite complementing genes are stably introduced in parallel into a complementing packaging cell line. However, this strategy requires chromosomal integration of a low copy number of viral genes and can be inefficient when multiple deletions must be complemented. Andrews J. L. et al. (5) showed that a vector deleted of E1, E2a, E3 and E4 region can not be propagated to high titer. Zhou H. et al. (54) demonstrated that multiple integrated copies of DBP gene are necessary in order to efficiently propagate an E1/E2a deleted vector at titers approaching those usually reached by first generation adenoviral vectors.

The development of efficient packaging/producer cell lines represents one of the most challenging tasks associated with the development of helper-independent adenoviral vectors. Therefore, an important requirement for the continued development and use of adenovirus-derived vectors is the design of helper independent producer cells lines that facilitate the production of high titer preparations of multi- or fully-deleted adenoviral vectors. An ideal solution would be development of a adenoviral vector system utilizing helper or producer cell lines that are amenable to high titer propagation of a fully deleted helper-independent adenoviral vector.

### SUMMARY OF THE INVENTION

The present invention provides an episomal plasmid, referred to herein as an adenoviral amplicon or replicon, which is capable of inducible self-replication in the nucleus of a mammalian cell. The disclosed adenoviral amplicon, is characterized by the following characteristics: (i) it contains the EBV latent origin of replication (oriP) and a human Ad5 inverted terminal repeats (ITRs) junction; and(ii) it inducibly expresses all three adenovirus type 5 early region 2(E2) genes as well as early region 4 (E4) ORF6 under the control of a Tet-dependent promoter. As shown herein, when the disclosed amplicon is used to transform 293EBNA cells expressing a Tet transcription silencer (tTS) and a reverse Tet transactivator (rtTA2) the resulting stable cell line (2E2), in the presence of doxycycline, produced higher levels of polymerase, precursor terminal protein (pTP) and DNA binding protein (DBP) than 293 cells infected with a first generation Ad vector. The data provided herein, further establish that use of the producer cell line (i.e. 2E2), disclosed herein can be used for the propagation of a multi-deleted ΔE1, E2, E3, E4 Ad vector. Accordingly, the disclosed Ad/EBV amplicon provides an important contribution towards the production of an efficient helper cell line that is suitable for high titer propagation of multi- or fully-deleted adenoviral vectors.

The first aspect of the present invention provides an adenoviral amplicon comprising: (a) an EBV-derived origin of replication (Ori-P) to promote maintenance of the amplicon within the nucleus of dividing cells expressing EBNA-1 protein; (b) an Ad5 origin of replication in form of Ad5 viral ITR junction which allows for amplication in an Ad-based manner; (c) a first transcriptional unit consisting of nucleic acid sequences encoding Ad5-derived polymerase and preterminal protein; (d) a second transcription unit consisting of a nucleic acid sequence encoding Ad5 DNA binding protein and E4 ORF6; and (e) a marker of selection; wherein the first and second transcriptional units are fused to a bi-directional tetracycline-dependent promoter. In a specific embodiment the invention provides the Ad5 E2/E4 ORF6 amplicon, pE2.

In an alternative aspect, the invention further provides an episomal plasmid comprising the nucleotide sequence of the plasmid deposited on Oct. 15, 2004 with the Belgian Coordinated Collections of Microorganisms Laboratory of Molecular Biology (BCCM/LMBP, Ghent University, Technologiepark 927, B-9052 Gent-Zwijnaarde, Belgium) Plasmid Collection as an original deposit under the Budapest Treaty. The deposit was assigned accession number LMBP 4972. This deposit will be maintained under the terms of the Budapest Treaty on the International Recognition of the Deposit of Microorganisms for the Purposes of Patent Procedure. This deposit was made merely as a convenience for those of skill in the art and are not an admission that a deposit is required under 35 U.S.C. § 112. All restrictions on the availability to the public of the deposited material will be irrevocably removed, except for the requirements specified in 37 C.F.R. §1.808(b), upon the granting of a patent.

The presence of EBV nuclear antigen-1 (EBNA-1) in combination with the OriP latent origin of replication, confer the functions of autonomous episomal replication and nuclear retention in a stable copy number, replicating only once per cell cycle (48). Because the coding sequences for the Ad5 polymerase, pTP and DBP responsible for adenovirus DNA replication, as well as E4orf6, are arranged into two bi-cistronic transcription units under Tet promoter control, when the Ad/EBV episome is transcriptionally silent, it is maintained as a latent viral element. As shown herein, the disclosed amplicon replicates upon induction of E2 gene expression, resulting in an increase in copy number.

In an alternative embodiment the invention contemplates Ad5 E2/E4ORF6 amplicons further comprising an expression cassette encoding a transgene of interest fused to a promoter. Transgenes of interest include human genes encoding proteins such as, but not limited to, immunoglobulins or fragments of immunoglobulins, single chain antibodies, bi-specific antibodies, erythropoietin, growth hormone, cytokines like 1-2 and IL-10-related cytokines, including IL-19, IL-20, IL-22, IL-24, IL-26, IL-28 and IL-29 genes; viral genes such as core, E1, E2 or the non structural region of HCV; HIV-1 gp41, GP120, gag, pol, nef of HIV, HSV-2 glycoprotein D; HPV L1, L2, E6 and E7 proteins, the spike (S) glycoprotein of the SARS-CoV; plasma membrane proteins such as viral receptors including the SARS-CoV ACE2 receptor, the HIV-1 receptor CD4 and chemokine co-receptors, The HCV receptors CD81, SRB 1, L-SIGN and heparin sulfated syndecans; G-protein coupled receptors (GPCRs), tyrosine-kinase cell surface receptors.

A second aspect of the present the invention provides a producer/helper cell line comprising an adenoviral amplicon of the invention. More specifically, the invention provides an adenoviral packaging cell line which expresses:(a) Ad5 E1 proteins; (b) an EBV-derived EBNA protein;(c) a Tet transcriptional silencer; (d) a Tet reverse transactivator; (e) an adenoviral amplicon consisting of: an EBV-derived oriP, an adenoviral ITR junction, and a first transcriptional unit consisting of nucleic acid sequences encoding Ad5-derived polymerase, preterminal protein in combination with a second transcription unit consisting of a nucleic acid sequence encoding Ad5 DNA binding protein and E4 ORF6, wherein the first and second transcriptional units are fused to a bi-directional tetracycline-dependent promoter; and(f)a selection marker.

In a particular embodiment, this aspect of the invention is exemplified herein by transforming 293EBNAtet cells (defined herein as 293EBNA cells expressing the Tet transcriptional silencer tTS^{kid} and the tet reverse transactivator rtTA2) with pE2, thereby producing a cell line suitable for use as a producer cell line for the propagation of a ΔE1,E2,E3,E4 Ad vectors. The packaging cell line exemplified herein is referred to as 2E2. The Ad5 ΔE1,E2,E3,E4 Ad vector of the disclosed system is characterized by a cloning capacity up to 12.4 Kb and by a reduced leakiness of viral gene expression. Producer cells subject of this invention are useful for, among other things, the production of recombinant adenoviruses designed for gene therapy and vaccination.

Another aspect of the present invention provides a method for producing replication-defective adenoviral vectors for use in therapeutic applications. For example, in a particular embodiment the invention provides immunogenic compositions for use as vaccines to induce an immunogenic response against antigens expressed by infectious agents/pathogens. In an alternative embodiment, the invention provides vaccines suitable for inducing an immune response against a tumor antigen. This aspect of the invention is exemplified herein by constructing a ΔE1-E4 expression vector expressing the entire HCV polyprotein and utilizing the vector in immunization experiments.

In one embodiment the invention provides a method for producing replication defective adenovirus comprising a transgene of interest, which comprises: introducing an multiply-deleted adenoviral expression vector into a packaging cell which expresses: an EBV-derived EBNA protein; a Tet transcriptional silencer; a Tet reverse transactivator; an adenoviral expression vector consisting of: an EBV-derived ori-P, an adenoviral ITR junction, and a first transcriptional unit consisting of nucleic acid sequences encoding Ad5 E2-derived polymerase, preterminal protein in combination with a second transcription unit consisting of a nucleic acid sequence encoding Ad5 DNA binding protein and E4 ORF6, wherein the first and second transcriptional units are fused to a bi-directional tetracycline-inducible promoter and an expression cassette encoding a transgene of interest fused to a promoter; inducing expression of the E2 and E40RF6 coding sequences; and harvesting the replication defection adenoviruses which are produced. In a particular embodiment, expression of the E2 and E40RF6 coding sequences is induced by contacting the packaging cells with doxycycline, which triggers the replication of the pE2 amplicon that is characterized by over-expression of the E2 and E40RF6 coding sequences.

In alternative embodiments, the method of the invention contemplates the use of 293EBNA cells expressing tTS^{kid} and rtTA2, as packaging cells for multi-deleted human Ad5 adenoviral vector lacking E1, E2, E3 and E4 genes. The invention further provides recombinant replication defective adenovirus particles harvested and purified according to the production methods disclosed and claimed herein.

Other features and advantages of the present invention are apparent from the disclosure provided herein. The examples illustrate different components and methodologies useful in practicing the claimed invention. It is to be understood, that the examples are not intended to be construed in a manner which limits the invention. Based on the present disclosure the skilled artisan can identify and employ other components and methodologies for practicing the present invention.

### BRIEF DESCRIPTION OF THE DRAWINGS

Text FIGS. 1A-1B provide schematic representations of the plasmid used to produce stable 293EBNATet clones. Panel A provides a linear representation of the plasmid components. Abbreviations include: reverse Tet trans-activator (rtTA); Tet silencer (tTS); ECMV internal ribosome entry site (IRES); intron sequence (intS); and puromycin resistance (Puro^{R}).

Panel B provides a schematic representation of pE2 plasmid. A head-to-tail junction of Ad5 inverted terminal repeats derived from pFG140 was cloned in the plasmid (ITRs, grey arrowheads). Ad5 early genes are indicated by black arrows: Polymerase (Pol), pre-Terminal Protein (pTP), DNA binding protein (DBP) and E4orf6 were inserted into two bicistronic expression cassettes driven by Tet responsive elements (TRE, white boxes); EBV latent origin of replication (OriP) flanked by chicken β-globin insulator sequences (HS4) are indicated by dotted box and grey boxes.

FIG. 2 provides a graphic representation of luciferase expression in AdTetLuc infected clones. 293EBNA cells and different 293EBNA/Tet clones were infected with AdTetLuc (m.o.i 10) in presence (black columns) or absence (white columns) of 1 μg/ml doxycycline. Luciferase activity in cell lysates was evaluated 48 hours post-infection.

FIG. 3 provides a schematic representation of pE2 in circular and linear form. DNA fragments obtained by NotI digestion allowing to differentiate between circular and linear form of pE2 are also indicated.

FIGS. 4A-4B. Panel A provides a Southern blot analysis demonstrating replication of pE2 upon activation of Ad5 E2 gene expression by doxycycline. 10⁸ copies of NotI-digested pE2 were loaded in the first lane. Episomal DNA extracted from 293 EBNA Tet cells 48 hours after transfection with pE2 without/with doxycycline and digested with NotI and DpnI was loaded in lanes 2 and 3. The 12.6 and 4.4 Kb bands, indicative of circular and linear monomeric forms, are indicated with black arrows. Size of DNA markers are indicated on the right of the figure (Kb).

Panel B provides a Western blot analysis demonstrating tet-inducible expression of E2 proteins. Western blots analysis of DBP, pTP and Polymerase protein in 293EBNATet cells transfected with pE2 amplicon with (+) (lanes 3, 6, and 9) or without (−) (lanes 2, 5, and 8) doxycycline. Negative (non transfected) controls are provided in lanes 1,4, and 7. E2 proteins were detected with specific rabbit antisera (polymerase, pTP) or mouse monoclonal antibody (DBP).

FIG. 5A-5B. Structure of pE2 Extracted from 2E2 Clone and Expression of E2 Proteins. Panel A is a Southern blot analysis elucidating the structure of pE2 extracted from clones 293EBNATet and 2E2. Southern blot analysis of DNA extracted from 293EBNATet and 2E2 clone. DNA extracted following the Hirt method was digested with BamHI, separated on 1% agarose gel transferred on nylon membrane and hybridized with pE2 DNA labeled with ³²P. pE2 vector was loaded in the first lane as reference; DNA extracted from 293EBNATet cells (negative control) and from 2E2 clone was loaded in the second and third lane respectively.

Panel B is a Western blot analysis demonstrating tet inducible expression of E2 proteins in 2E2 stable cell line compared to E2 expression in cells infected with Ad5ΔE1 vector. E2a and E2b protein expression by 2E2 clone was evaluated by Western blot in presence (+) (lane 3) and in absence (−) (lane 2)of doxycycline (1 μg/ml) (lanes 2, 3; 5,6; 8,9) and compared to the expression levels of E2 proteins from non induced 2E2 cells infected with an m.o.i. of 500 of a FG Ad5ΔE1 vector (lane 1). Migration of molecular weight markers (kDa) is indicated on the left of the figure.

FIG. 6 provides a series of photographs illustrating the use of 2E2 cells to rescue and propagate an Ad ΔE₁₋₄ vector expressing EGFP. Ad5ΔE₁₋₄EGFP virus amplification in 2E2 clone with silenced (−doxy) or activated (+doxy) E2/orf6 gene expression. P0=transfection, P1 and P2 were obtained by infecting cells with 1/10 of total crude lysate from previous infection passage.

FIGS. 7A-7B. Panel A provides a schematic map of Ad5 virus. All deleted regions are indicated in the diagram. E1, E2a, E3 and six of the seven E4 orfs with the exception of orf3 were completely deleted from the vector backbone. The deletions of polymerase and pre-terminal protein were only partials. The E1 region is replaced with a HCV polyprotein expression cassette driven by MCMV promoter. HindIII restriction sites used in vector genome restriction analysis are indicated (▮).

Panel B provides schematic representation of the HCV (strain BK) polyprotein expression cassette that was introduced in the E1 region of the multiply-deleted vector. HCV 5′ and 3′ UTR sequences were eliminated; an optimized Kozak sequence was fused to the 5′ of the polyprotein. Expression is regulated by mouseCMV promoter (mCMV) and bovine growth hormone polyA (BGH polyA).

FIG. 8 provides a restriction analysis of Ad ΔE₁₋₄orf3⁺HCV. Viral DNA extracted from CsCl-purified viral particles and plasmid DNAs were digested with HindIII and end-labeled with (³³P)dATP by fill-in reaction with Klenow enzyme. The viral DNA restriction pattern of purified Ad ΔE₁₋₄orf3⁺HCV vector (lane 4) was compared to the original plasmid (lane 3). FG (ΔE1-E3) Ad5 (lane 1) and Ad5ΔE₁₋₄orf3⁺ empty vector (lane 2) backbones restricted with HindIII were included in the gel. a,b,c,d indicates multiply deleted vector DNA bands containing deletions and the corresponding bands in the FG (ΔE1-E3) Ad5 pattern.

FIG. 9 provides a Western blot analysis demonstrating expression of HCV proteins in Ad5ΔE₁₋₄HCV infected cells. HeLa cells were infected with Ad5ΔE₁₋₄HCV with an m.o.i. of 10. HCV proteins were detected in cell extracts by Western blot analysis with HCV-specific antibodies. Lysates from HeLa cells, prepared 48 hours post-infection were loaded in lanes 3 and compared to lysate from uninfected control cells (lane 1); and lysate from HeLa cells transfected with mCMV-HCV vector DNA (lane 2). Specific bands are indicated by arrows.

FIG. 10 provides graphic representation of FACS data characterizing the in vivo CD8+ T cell response to Ad5ΔE₁₋₄HCV virus immunization in mice. A2.1 (a) and CB6F1 (b). Freshly isolated splenocytes of mice immunized i.m. with 10¹⁰ vp were tested for CD8+ T cell response to pools of HCV-peptides by 3 weeks later intracellular staining for IFN-γ. x-axis anti-INF-γ, y-axis anti-CD8. poolC (Core), pool F-G (NS3), pool H(NS4), pool I-L-M (NS5a/b).

FIG. 11 provides the nucleotide and/or amino acid sequences of the polynucleotide and polypeptide sequences (i.e., SEQ ID NOS.: 1-21) described in this disclosure.

FIGS. 12A-12C show the modality followed in mapping the epitope within NS3 helicase. Splenocytes purified from two mice (mouse 4, solid bar, mouse 5, stipled bar) primed with Ad5Δ_{E1-E4}HCV and boosted with pSh-Ad5-HCV were tested in γ-IFN-Elispot on a two dimensional sub-set of peptides (from 1 to XVIII) covering the entire NS3 helicase region (FIG. 12A). The data provided is FIG. 12B summarizes the points of intersection between the peptide pools which elicited a response above the positivity threshold identified in Panel A used to characterize the immune response. Panel 12C summarizes the results of a γ-IFN-Elispot Assay performed to identify the NS3 epitope responsible for the response.

FIG. 13 shows the efficacy of the immunization in inducing protection to VV-NS challenge. Grey dots represent geometric mean titres (N=5). The asterisk indicates p<0.05 respect to the control (Mann-Whitney rank).

FIGS. 14A-B summarize immune responses elicited in rhesus monkeys in response to Ad5Δ_{E1-E4}-HCV immunization. Panel A represents the immune response over the time elicited in monkey 4061 upon one administration of Ad5Δ_{E1-E4}-HCV and analysed by γ-IFN-Elispot. Results are expressed as γ-IFN spot forming cells (SFC) per 10⁶ PBMC. Each bar represents the response to a separate peptide pool.

Panel B shows the immune response induced in three individual monkeys by one administration of Ad5Δ_{E1-E4}-HCV and analysed by γ-IFN-Elispot 6 weeks post-injection. Results are expressed as γ-IFN spot forming cells (SFC) per 10⁶ PBMC. Each bar represents the response to a separate peptide pool.

### DETAILED DESCRIPTION OF THE INVENTION

The numerical citations included at the end of particular sentences refer to the numbered list of references included at the end of the specification. The references cited herein are not admitted to be prior art to the invention.

It is important to an understanding of the present invention to note that all technical and scientific terms used herein, unless otherwise defined, are intended to have the same meaning as commonly understood by one of ordinary skill in the art. Certain terms that are set forth below, or may be defined in this description when they are used for the first time.

As used herein the term “amplicon” refers to an episome or an extrachromosomal DNA element which is capable of replicating when essential gene functions are provided. Generally speaking, an adenoviral amplicon is understood to include at least a portion of each terminal repeat required to support the replication of the viral DNA. Eukaryotic viral amplicons preferably comprise at least about 90% of the full ITR sequences. Accordingly, an “adenoviral amplicon” comprises an ITR junction and any suitable origin of replication.

As used herein the term “transfection” means any suitable method of transferring a DNA from the outside of a cell to the inside of a cell so that the cell remains biologically viable. As used herein the term includes the introduction of DNA into a host cell by any means, including without limitation transfection of episomes and other circular or linear DNA forms. This includes methods of gene therapy, such as those described herein. Any appropriate transfection method can be used to practice the invention, including without limitation calcium phosphate co-precipitation, electroporation, gene gun transfection, lipofection or other cationic lipid based transfection. These techniques are well known to those of ordinary skill in the art.

The terms used herein are not intended to be limiting of the invention. For example, the term “gene” includes cDNAs, RNA, or other polynucleotides that encode gene products. In using the terms “nucleic acid”, “RNA”, “DNA”, etc., we do not mean to limit the chemical structures that can be used in particular steps For example, it is well known to those skilled in the art that RNA can generally be substituted for DNA, and as such, the use of the term “DNA” should be read to include this substitution. In addition, it is known that a variety of nucleic acid analogues and derivatives is also within the scope of the present invention. “Expression” of a gene or nucleic acid encompasses not only cellular gene expression, but also the transcription and translation of nucleic acid(s) in cloning systems and in any other context.

It is appreciated that the massive production of adenovirus from a natural infection is the consequence of a coordination between viral DNA replication and major late promoter activity. In practice, this strategy leads to the accumulation of a high copy number of transcriptionally active templates, which has the effect of generating a large pool of structural proteins which are required to package the virions. Although the prior art includes several helper cell lines expressing one or more viral proteins for the production of adenoviral vectors that are defective for one or more viral proteins, a production system characterized by a coordinated series of events (e.g. viral DNA replication and expression of the requisite structural proteins) which mimics a natural infection has not previously been described. Rather than utilize a strategy that calls for the production of a complementary helper cell line based on integration of the complementing genes into host cell chromosomes, an episomal plasmid, carrying all of the requisite nonstructural E2 genes required for adenoviral replication, which is capable of self-replication in the host cell has been produced.

In response to the art recognized limitations associated with using helper virus-dependent production systems, the instant invention provides a novel adenoviral amplicon that can be used to create producer cell lines that are capable of complementing multi- or fully-deleted adenoviral vectors. The amplicon has been designed to function in a manner which mimics the stages of a natural adenoviral infection, thereby maximizing the efficiency of helper virus-independent vector production. As shown herein, this is accomplished by engineering packaging cells in which the disclosed episome (i.e., adenoviral amplicon) is maintained in a latent phase in the nucleus of the packaging cell line by actively suppressing/delaying expression of the adenoviral early genes required to initiate the viral transcriptional cascade.

The latency is achieved by exploiting the nuclear retention features of the Epstein-Bar virus (EBV)-derived DNA replicative elements and the use of an inducible expression system (exemplified herein by the tetracycline regulatory expression system). Upon induction, a replicative phase is activated which results in transcription of the episomal sequences resulting in expression of the adenoviral E2 genes required for replication (i.e., polymerase, pre-terminal protein and DNA binding protein). In practice, the outcome of the transition from the latent to the replicative phase, facilitates the accumulation of large amounts of the complementing viral proteins required to efficiently package a multi- or fully-deleted adenoviral vector comprising a transgene. Accordingly, the amplicon is designed to allow the packaging cell to function in a manner that mimics the series of events which typically produces high titer virion production during the late phase of a natural infection. Accordingly, the disclosed amplicon and packaging cell line enables an efficient high titer method of producing helper virus-independent pharmaceutical grade vectors.

The episomal plasmid (pE2) is characterized by the following features it comprises: (i) an element, such as the EBV plasmid origin of replication, which renders the episome capable of autonomous replication and maintains the episome in multiple copies by promoting nuclear retention, (ii) an Ad5 inverted terminal repeat (ITRs) junction which allows DNA replication in linear form; and (iii), it mediates the inducible expression of E2 adenoviral genes necessary for adenoviral replication (e.g., polymerase, pre-terminal protein and DNA binding protein, as well as early region 4 (E4) ORF6.

Those skilled in the art will appreciate that for viral DNA replication only two regions of the Ad5 viral DNA (disclosed in GenBank BK000408) are known to be required in cis. These are the left inverted terminal repeat, or ITR, (bp 1 to approximately 103 of Ad5) and the right ITR (bp 35833 to 35935 of Ad5). The presence of an origin of replication system derived from EBV allows the amplicon to be retained in the nucleus in multiple copies, replicating in synchrony with the chromosomal DNA, while the presence of the adenoviral ITR junction allows the amplicon to replicate at a high copy number in the presence of proteins coded by the E2 regions. The use of an inducible promoter on the amplicon places the adenoviral genes, required for the replication of the episome, as well as for the propagation of a multi- of fully-deleted adenoviral vector comprising a transgene, under the control of a strictly regulation-responsive inducible promoter. Generally speaking, an inducible promoter is a promoter that is induced by an activator. In the absence of the inductor acting on the inducible promoter, the adenoviral genes contained on the episome are not expressed, and there is no production of viral protein and minimal risk of viral protein-induced cytotoxicity.

In practice, the disclosed amplicon (e.g., episome) may comprise elements which may work in concert with other elements (for example an activating factor) present in the host cell to simultaneously fulfill one or more of the above mentioned characteristics. For example, the same element (DNA sequence) may confer the capability of self-replication and promote nuclear retention. It is to be understood, that DNA sequences derived from alternative viral replication systems, can also be used to practice the invention. For example, the origins of replication and activating factors derived from bovine papilloma virus (BPV) (60), or sequences derived from vectors based on SV40 origin-T antigen system provide suitable alternatives.

Although the examples herein describe the use of host cells expressing EBNA-1, it will be readily apparent that an alternative activating factor can optionally be introduced into host cells, by including a coding sequence on either the same episomal unit (amplicon) which carries the adenoviral genes, on a second genetic unit that is capable of replication, or by stable integration into the host cell genome. For example, instead of employing an EBNA1 antigen and EBV origin of replication, as used herein, it is possible to employ bovine papilloma virus (BPV) E1 and E2 antigens in combination with the BPV origin of replication. The E1 antigen is a helicase required for initiation of replication and elongation while the E2 antigen is a transcription factor that assists binding of the E1 antigen to the origin of replication (61). Together these viral proteins are also known to promote nuclear retention of an episome in cells that are competent for appropriate transfection.

Defined genetic elements of the EBV genome are known to stably maintain non-integrating, autonomously replicating episomal vectors in primate cells and to support stable replication of the plasmid. The requisite genetic elements include the cis acting origin of plasmid replication (oriP) and the trans acting Epstein-Barr nuclear antigen (EBNA-1) protein. More specifically, the EBV-derived elements, oriP and EBNA-1, have been used to support stable replication of recombinant episomes, which are found exclusively as unintegrated extrachromosomal molecules at a number ranging from 1 to 90, in mammalian cells transfected with these vectors.

Plasmids containing the replication origin oriP of the EBV genome and allowing the expression of the EBNA1 viral protein (641 amino acids) are maintained in a stable episomal manner in the transfected human cells and their replication is synchronous with cell division. As shown herein, the EBV origin of replication (OriP)) used in the presence of the activating factor EBNA-1 confers the capability of self-replication and promote nuclear retention. While not wishing to be bound by theory, it is thought that the EBNA1 protein attaches to the 30 bp repeats at the level of the replication origin and allows the recruitment of cellular factors at the time of the S phase and the replication, synchronously with cell division, of a plasmid having the oriP sequence in cis. Furthermore, EBNA1, probably through the simultaneous attachment at the level of the repeat units and of chromosomal structures, allows intranuclear maintenance and the segregation of the episome at the time of cell division. These elements allow, on their own, at the time of replication, episomal maintenance and segregation of multiple copies per cell of a plasmid vector.

Any suitable EBV origin of replication DNA sequence can be employed in the episomes used in the present invention. An example of a suitable EBV origin of replication sequence (oriP) is disclosed in GenBank V01555. The oriP region spans the sequence from nucleotide 7333 to nucleotide 9312 in this GenBank sequence. The oriP sequence utilized in the episomes described herein is composed of a repetition of 20 units of 30 bp, separated by 960 bp from the replication origin which is formed by an inverted repeat unit of 65 bp and comprises 4 imperfect copies of the 30 bp unit.

Epstein-Barr virus (EBV)-derived oriP is composed of two clusters of EBNA-1 binding sequences: a family of repeat and a dyad symmetry sequence. Both elements have multiple binding sites for EBNA-1 and are essential for replication and nuclear retention of plasmids containing oriP. Host cells factors are believed to assist the replication and nuclear retention of the episomes disclosed herein. Generally speaking, a suitable oriP sequence includes the family of repeats and the region of dyad symmetry known to be required for oriP function. EBV oriP sequences that can be used in the invention include those containing modifications from naturally occurring sequences, such as those containing deletions, insertions, substitutions and duplications, of native sequences. Such derivative sequences are obtainable, for example, by maintaining the known regions described above that are required for oriP function. Also, conservative substitutions are well known and available to those in the art. The oriP sequence employed is one that functions effectively in the host cell to direct the replication of the episome in which the oriP sequence is found in the presence of a sufficiently high amount of an EBNA1 protein.

DNA encoding any suitable EBNA 1 protein can be expressed by the producer cells of the invention. EBNA1-encoding DNA is commercially available from Invitrogen, and is contained in several of its EBV series plasmids. Furthermore, DNA encoding the EBNA protein can encode variants of the naturally occurring EBNA 1 amino acid sequence, including those containing, e.g., deletions, additions, insertions, or substitutions, wherein the expressed protein supports replication of EBV oriP-containing episomes in the host cell. This includes, as with other sequences described herein, functionally conservative nucleic acid sequences encoding amino acid sequences conservative variants, sequences having greater than 90%, preferably greater than 95%, identity or homology as determined by BLAST or FASTA algorithms and sequences hybridizing under high stringency hybridization conditions. Furthermore, degenerate DNA sequences that encode the same EBNA1 protein can be employed. Degenerate DNA sequences capable of expressing the same amino acid sequence are well known in the art, as are methods of constructing and expressing such DNA sequences.

EBNA1 can be stably transfected into any primate or canine cell using well known techniques, and the resulting cell line that expresses EBNA1 from an integrated gene copy can be used to create a suitable production cell line. Alternately, a cell line that already harbors infectious or defective EBV can be used, as long as EBNA1 is expressed. This includes many EBV transformed lymphoblasts available from the ATCC. As discussed above, it is also possible to express EBNA1 from a stably transfected episome. Transfection of cell lines that already express EBNA1 can be extremely advantageous, as the ability of such cells to stably maintain episomal constructs can be enhanced by several orders of magnitude and stable cell lines can be generated in as little as two to three weeks (62). These methods, however, require the additional step of producing a cell line which constitutively expresses EBNA1 from an integrated gene.

As shown herein, the tetracycline promoter, which is responsive to tetracycline or one of its common analogs, such as doxycycline (Dox), is suitable for use in the disclosed episomal units (e.g., amplicons or replicons). Doxycycline, an analog of tetracycline, is widely accepted because of its safe use in humans, its specificity for the bacterial tetracycline repressor (TetR). Briefly, the tetracycline-dependent regulatory system (tet system) is based upon the interaction between the tetracycline transactivator (tTA), consisting of the procaryotic TetR fused to the activator domain of the herpes simplex virus VP16 protein, and the tetracycline-responsive element (TRE), consisting of seven copies of the procaryotic tetracycline operator site (tetO) fused to a minimal CMV promoter (68). In the presence of tetracycline (tet), tTA loses its ability to bind TRE and the expression is shut off. A reverse transactivator (rtTA) has been derived from tTA by mutagenesis. In contrast to tTA, rtTA only binds TRE in the presence of tet.

In order to obtain a stringent control of gene expression by reducing the basal level of transcription, we used the Tet regulatory system exploiting the combination of Tet transcriptional silencer, tTS^{kid}, (16) with the new improved version of reverse tet transactivator recently described (29). tTS^{kid} contains the KRAB domain of the kidney protein Kid-1 that is known to function as a repressor of transcription. tTS^{kid} binds to the Tet promoter in absence of the effector drug thus reducing the basal level of transcription. The combination with the reverse Tet transactivator allows the construction of an activating/repressing system regulated by doxycycline addition. To this end, the two genes were combined in a bicistronic transcription unit by using EMCV IRES as described in FIG. 1A.

Several promoter systems are available which are capable of directing inducible gene expression in eukaryotic cells. These include promoters whose activity is modified in response to heavy-metal ions, (63), (64), isopropyl-beta-D-thiogalactoside (65), hormones such as corticosteroids (66) progesterone antagonists (67) or tetracycline (68). However, other well-known inducible regulatory elements which are responsive to activators such as ecdysone, rapamycin, RU486, dexamthasone and heavy metals (i.e., Zn or Cd) are also suitable. It is well within the capabilities of a skilled artisan to adapt an alternative regulatory element for use in the present invention. For the purpose of the present invention, any regulatory element can be utilized, provided that it ensures a sufficient level or regulatory control and is inducible by an activator that is acceptable for pharmacological use. Further, in order to facilitate tight regulation of gene expression, it is to be understood that inducible promoter can also be operatively linked to other regulatory elements, such as a tetracycline-responsive transactivator and/or silencer (rtTA and tTs).

All of the expression cassettes (defined as comprising a transgene of interest and the requisite regulatory sequences to direct expression in a mammalian cell disclosed herein) were constructed in the context of an Ad-shuttle vector that contains in addition to CMV promoter and BGH polyA signal for transgene expression, the Ad5 sequences [nt] 1-450 (left) (SEQ ID NO: 1) and [nt] 3511-5792 (right) (SEQ ID NO: 2) to allow the insertion in the E1 region of pAd5ΔE₁₋₄orf3⁺ by homologous recombination in E. Coli BJ5183.EGFP cDNA was obtained from pEGFP plasmid (Clontech) then cloned in Ad-shuttle plasmid obtaining pShAd5 EGFP.

In the methods described herein, a conventional selection marker is used to select for cells that have been successfully transfected with an episome encoding the desired sequences. Such selection normally involves exposing transfected cells to antibiotics or other substances that initiate the relevant selection process. Selectable marker genes for use in the episomes employed in the invention are genes that encode proteins conferring resistance to specific antibiotics and/or factors that allow cells harboring these genes to grow in the presence of the cognate antibiotics or factors. Non-limiting examples of eukaryotic selectable markers include antibiotic resistance genes conferring resistance to hygromycin (hyg or hph, commercially available from Life Technologies, Inc.; Gaithesboro, Md.); neomycin (neo, commercially available from Life Technologies, Inc. Gaithesboro, Md.); zeocin (Sh Ble, commercially available from Pharmingen, San Diego Calif.); puromycin (pac, puromycin-N-acetyl-transferase, available from Clontech, Palo Alto Calif.), ouabain (oua, available from Pharmingen) and blasticidin (available from Invitrogen).

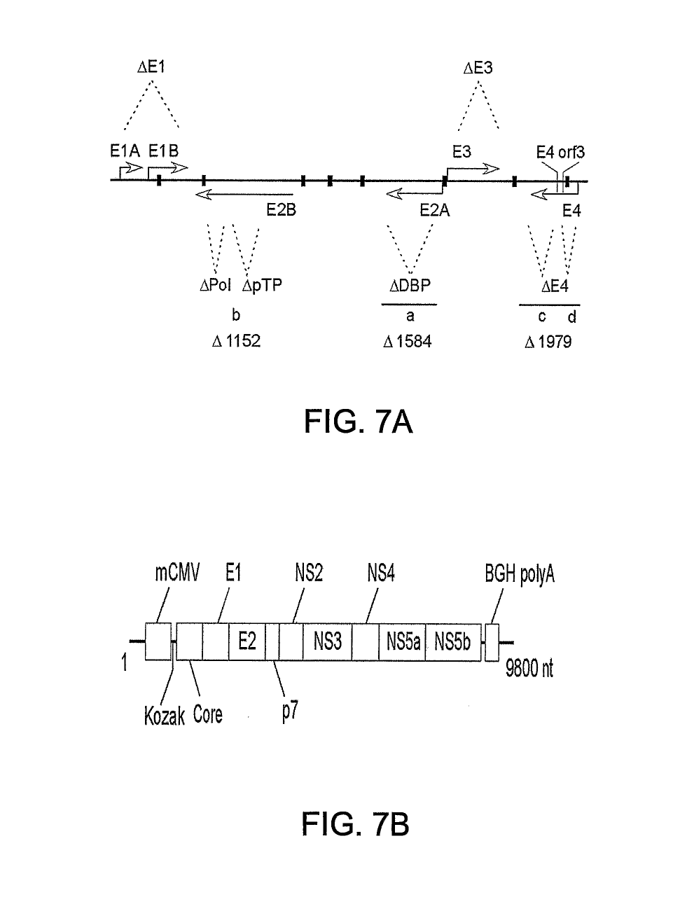

A schematic representation of the multiply deleted human Ad5vector backbone is shown in FIG. 7A. Besides the classical deletion of E1 and E3 regions (reviewed in 11), we have removed the entire coding sequence of DNA binding protein ([nt] 22245-24029; (SEQ ID NO:3) 1784 bp deletion) without affecting any other functions encoded in the r-strand, which encompasses the L4 intron. Portions of Polymerase ([nt] 7274-7883; (SEQ ID NO: 4) 609 bp deletion) and Pre-terminal protein (Ad5 [nt] 8919-9462 (SEQ ID NO: 5) 543 bp deletion) genes corresponding to the introns of tripartite leader sequence and major late units were deleted to knockout E2b gene expression. Furthermore, to prevent the production of a truncated non-active form of polymerase, the ATG start codon was mutated to CTG. The E4 region was totally deleted ([nt] 32830-34316 (SEQ ID NO: 6) and 34895-35443; (SEQ ID NO: 7) with the exception of orf3 that was directly fused to E4 promoter.

The theoretical space created in the Ad5 backbone by combining the deletion of all early genes is about 12.4 Kb. The large capacity of the new vector system was exploited to insert an expression cassette for the entire HCV polyprotein gene fused to the mouse cytomegalovirus (MCMV) promoter. The HCV polyprotein expression cassette was constructed by eliminating the 5′ and 3′ untranslated region, by inserting an optimal Kozak sequence upstream core ATG and by mutating the catalytic domain of NS5B replicase to eliminate the enzymatic activity (32). In order to increase the efficiency of transgene expression we substituted the human CMV promoter with mouse CMV promoter that was reported to be 4- to 30-fold more potent in FG adenoviral vectors (1).

FIG. 7B provides schematic representation of the HCV (strain BK) polyprotein expression cassette that was introduced in the E1 region of the multiply-deleted vector. HCV 5′ and 3′ UTR sequences were eliminated; an optimized Kozak sequence was fused to the 5′ of the polyprotein. Expression is regulated by mouseCMV promoter (mCMV) and bovine growth hormone polyA (BGH polyA).

It is known that maintenance of open reading frame 3 is required for the persistent expression in vivo and in vitro of transgenes regulated by an internal CMV promoter (18, 34). Thus, in addition to the 5700 bp deletion of a ΔE1E3 FG vector and accordingly to the packaging capacity of genome size of 105% of that of the wt (6), the new Ad5ΔE₁₋₄orf3⁺ viral vector can accommodate transgenes up to 12.4 Kb. To this end it is envisioned that defective adenoviral vectors comprising numerous transgenes of interest can be produced using the adenoviral amplicons and producer cells, and methods of the invention.

Suitable transgenes for use in the multi-deleted Ad5 viral backbone disclosed herein include but are not limited to the nucleic acid sequence encoding the immunogen (i.e., the transgene) that may be codon optimized for expression in a particular mammalian species. In one embodiment the invention provides an immunogenic composition (e.g., a vaccine) for inducing an immune response against antigens expressed by an infectious agent. For example it is desirable to elicit an immune response against a virus infecting humans and/or non-human animal species.

The multi-deleted Ad5 vector may also suitable to stimulate an immune response in humans or animals against proteins expressed by pathogens including bacteria, fungi, parasites. Staphylococcus aureus, streptococcus pyogenes, streptococcus pneumoniae, vibrio cholerae, clostridium tetani, neisseria meningitis, corynebacterium diphteriae, mycobacteria tuberculosis and leprae, listeria monocytogenes, legionella pneumofila are examples of bacteria against which but not limited to eliciting an immune response may be desirable. Examples of fungi and parasites can be: candida albicans, aspergillus fumigatus, histoplasma capsulatum, Plasmodium malariae, Leishmania major, trypanosome cruzi and brucei, Schistosoma haematobium, mansoni and japonicum; Entamoeba histolytica, different species of Filaria responsible for human filariasis.

Examples of virus families against which a prophylactic and/or therapeutic immune response would be desirable include the Picornaviridae family which includes six different genera such as Aphtovirus, Cardiovirus, Enterovirus, Hepatovirus, Parechovirus, Rhinovirus. All of them contain viruses infecting vertebrates. Examples of Picornavirus against which an immuneresponse would be desirable are: Foot-and-mouth disease viruses, Encephalomyocarditis viruses, Polioviruses, Coxackieviruses, Human hepatitis A virus, Human parechoviruses, Rhinoviruses. Caliciviridae family includes different genera associated with epidemic gastroenteritis in humans caused by the Norwalk group of viruses and other syndromes in animals like the hemorrhagic disease in rabbits associated with rabbit hemorrhagic disease virus or respiratory disease in cats caused by feline calicivirus. Another family is the Astroviridae which comprises viruses isolated y humans as well as many different animal species. Human astroviruses are associated with gastroenteritis and young children diarrhea. The Togaviridae family comprises two genera: alphavirus and rubivirus. Alphaviruses are associated with human and veterinary diseases such as arthritis (i.e. Chikungunya virus, Sindbis virus) or encephalitis (i.e. Eastern Equine Encephalitis Virus, Western Equine Encephalitis Virus). Rubella virus is the only member of the Rubivirus genus is responsible for outbreaks of a mild exanthematic disease associated with fever and lymphoadenopathy. Rubella virus infection is also associated with fetus abnormalities when acquired by mother during in early pregnancy. Flaviviridae is an other virus family consisting of three genera: the flaviviruses, the pestiviruses and the hepaciviruses that includes important human as well as animal pathogens. Many of the flavivirus genus members are arthropod-borne human pathogens causing a variety of diseases including fever, encephalitis and hemorrhagic fevers. Dengue Fever Viruses, Yellow Fever Virus, Japanese Encephalitis Virus, Wst Nile Fever Virus, Tick-borne Encephalitis Virus are pathogens of major global concern or of regional (endemic) concern. Pestivirus genus includes animal pathogens of major economic importance such as Bovine Viral Diarrhea Virus, Classical Swine Fever Virus, Border Disease Virus. Hepatitis C Virus is the only member of the Hepacivirus genus responsible for acute and chronic hepatitis. HCV proteins expressed by a recombinant adenovirus can elicit a protective as well as therapeutic immune response limiting the consequences of a viral infection affecting 170 million people worldwide.

Antigens derived from members of the Coronaviridae family can be expressed by recombinant adenovirus vectors in order to obtain protection against infection. Protection against the severe acute respiratory syndrome coronavirus (SARS-Co Virus) can be obtained by immunizing with the multi-deleted Ad5 vector expressing combinations of SARS-CoV proteins including without limitations nucleocapsid (N) protein, polymerase (P) protein, membrane (M) glycoprotein, spike (S) glycoprotein, small envelope (E) protein or any other polypeptide expressed by the virus. Rhabdoviridae family members including rabies virus can be target of recombinant vaccine expressing viral proteins. Other possible targets include the Filoviridae family comprising Ebola-like viruses and Marburg-like viruses genera, that is responsible of outbreaks of severe hemorrhagic fever; the Paramyxoviridae family comprising some of the most prevalent virus known in humans like measles, respiratory syncytial, parainfluenza viruses and viruses of veterinary interest like Newcastle disease and rinderpest viruses; the Orthomyxoviridae family including Influenza A,B,C viruses; Bunyaviridae family mainly transmitted by arthropod to vertebrate hosts comprising important human pathogens like Rift valley fever, Sin Nombre, Hantaan, Puumala viruses; Arenaviridae family comprising Lymphocytic choriomeningitis, Lassa fever, Argentine Hemorragic fever, bolivian Hemorragic fever viruses; Bornaviridae family comprising viruses causing central nervous system diseases mainly in horses and sheep; Reoviridae family including rotaviruses, the most important cause of severe diarrheal illness in infants and young children worldwide, orbiviruses that can affect both humans and other mammals (bluetongue, epizootic hemorrhagic disease viruses).

Suitable transgenes encoding viral antigens may also be obtained from members of the Retroviridae family, a large group of viruses comprising important human pathogens like human immunodeficiency virus 1 and 2 (HIV-1 and HIV-2) and human t-cell leukemia virus type 1 and 2 (HTLV 1 and 2) as well as non-human lentivirus such as Maedi/Visna viruses affecting sheep and goats, Equine infectious anemia virus affecting horses, bovine immunodeficiency virus affecting cattle, feline immunodeficiency virus affecting cats; Polyomaviridae family groups small DNA oncogenic viruses, prototype viruses are polyoma and SV40 infecting mouse and rhesus monkey respectively, (BK and JC viruses closely related to SV40 were isolated from human patients).

The Papillomaviridae family consists of a group of DNA viruses infecting higher vertebrates including humans generating warts.and condylomas. Infection of papilloma viruses was associated to cancer development in both humans and animals. Human papilloma viruses are associated with cervical cancer, vaginal cancer and skin cancer. The herpesviridae famils includes subfamilies in which are classified a number of important pathogens for humans and other mammals. Alternative sources of antigens include, but are not limited to herpes simplex viruses 1 and 2, varicella-zoster virus, Epstein-Barr virus, Cytomegalovirus, human herpesviruses 6A,6B and 7, Kaposi's sarcoma-associated herpesvirus. Further suitable source of antigens are members of the Poxyiridae family like monkeypox virus, molluscum contagiusum virus, smallpox virus; hepatitis B virus, the prototype member of the hepadnaviridae family as well as other virus causing acute and/or chronic hepatitis like hepatitis delta virus, hepatitis E virus.

In a second embodiment the invention provides an immunogenic composition (e.g., a vaccine) for inducing an immune response against a tumor antigen. A suitable composition would contain a recombinant chimpanzee adenovirus comprising an optimized nucleic acid sequence encoding a tumor antigen and a physiologically acceptable carrier. In particular embodiments, the coding sequence element of the cassette may encode a single immunogen, such as an antigen from a pathologic agent or a self-antigen, such as a tumor-associated antigen. In other embodiments, the coding sequence may encode more than one immunogen. For example, it may encode a combination of self-antigens such as: Her2 Neu, CEA, Hepcam, PSA, PSMA, Telomerase, gp100, Melan-A/MART-1, Muc-1, NY-ESO-1, Survivin, Stromelysin 3, Tyrosinase, MAGE3, CML68, CML66, OY-TES-1, SSX-2, SART-1, SART-2, SART-3, NY-CO-58, NY-BR-62, hKLP2, VEGF, 5T4.

The transcriptional promoter used to direct expression of the transgene is preferably recognized by an eukaryotic RNA polymerase. In a preferred embodiment, the promoter is a “strong” or “efficient” promoter, such the mouseCMV promoter (mCMV) used in the examples presented herein. An example of another strong promoter is the immediate early human cytomegalovirus promoter (Chapman et al, 1991 Nucl. Acids Res 19:3979-3986, which is incorporated by reference), preferably without intronic sequences. Thus, one alternative promoter suitable for use in the episomes disclosed and claimed herein includes a human CMV promoter. Those skilled in the art will appreciate that any of a number of other known promoters, such as the strong immunoglobulin, or other early or late viral promoters, such as, e.g, SV40 early or late promoters, Rous Sarcoma Virus (RSV) early promoters; eukaryotic cell promoters, such as, e.g., beta actin promoter (Ng, S.Y., Nuc. Acid Res. 17:601-615, 1989, Quitsche et al., J. Biol. Chem. 264:9539-9545, 1989), GADPH promoter (Alexander et al., Proc. Nat. Acad. Sci. USA 85:5092-5096, 1988, Ercolani et al., J. Biol. Chem. 263:15335-15341, 1988), metallothionein promoter (Karin et al. Cell 36: 371-379, 1989; Richards et al., Cell 37: 263-272, 1984); and concatenated response element promoters, such as cyclic AMP response element promoters (cre), serum response element promoter (sre), phorbol ester promoter (TPA) and response element promoters (tre) near a minimal TATA box.

Preferred transcription termination sequences present within the gene expression cassette are the bovine growth hormone terminator/polyadenylation signal (bGHpA). Alternative transcription termination/polyadenylation sequences include without limitation those derived from the thymidine kinase (tk) gene or SV40-derived sequences, such as found, e.g., in the pCEP4 vector (Invitrogen).

Having generally described the purposes, advantages, applications and methodology of this invention, the following non-limiting examples are provided to describe in a detailed fashion, various embodiments of this invention. However, it should be appreciated that the invention described herein is not limited to the specifics of the following examples, which are provided merely as a guide for those wishing to practice this invention. The scope of the invention is to be evaluated with reference to the complete disclosure and the claims appended hereto.

Materials and Methods

The practice of the present invention will employ, unless otherwise indicated, conventional techniques of molecular biology (including recombinant techniques), microbiology, cell biology, and biochemistry, which are within the skill of the art. Such techniques are explained fully in reference such as, “Molecular Cloning: A Laboratory Manual”, 2nd edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4th edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

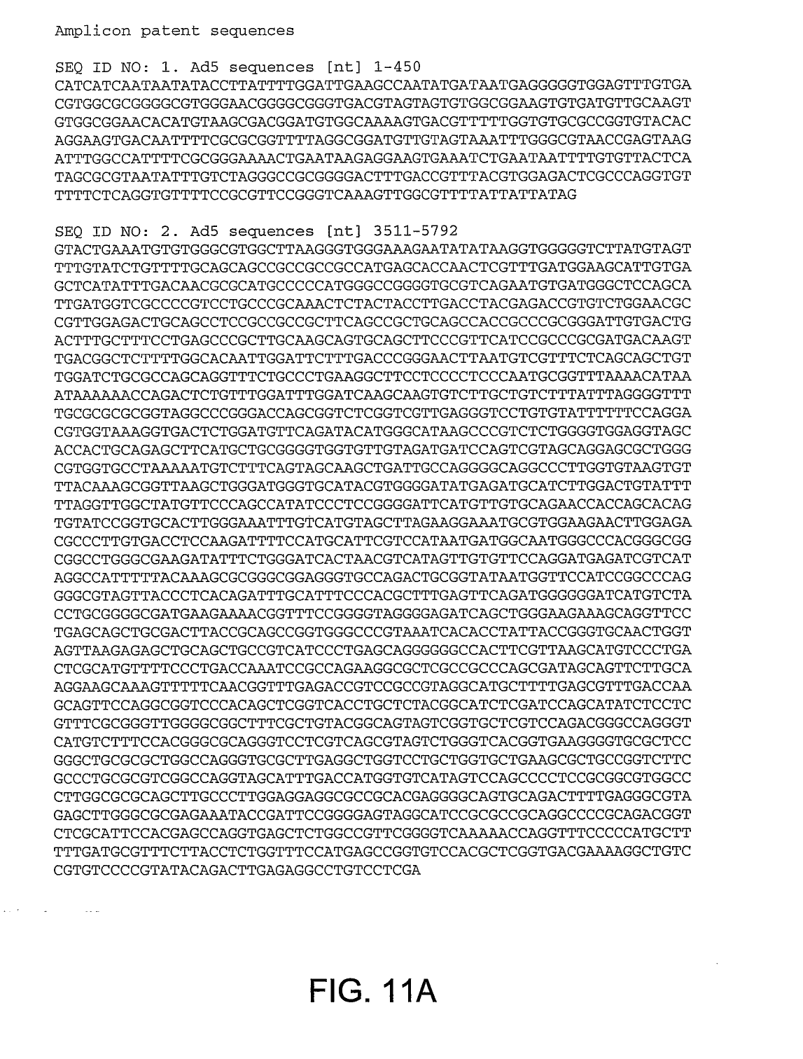

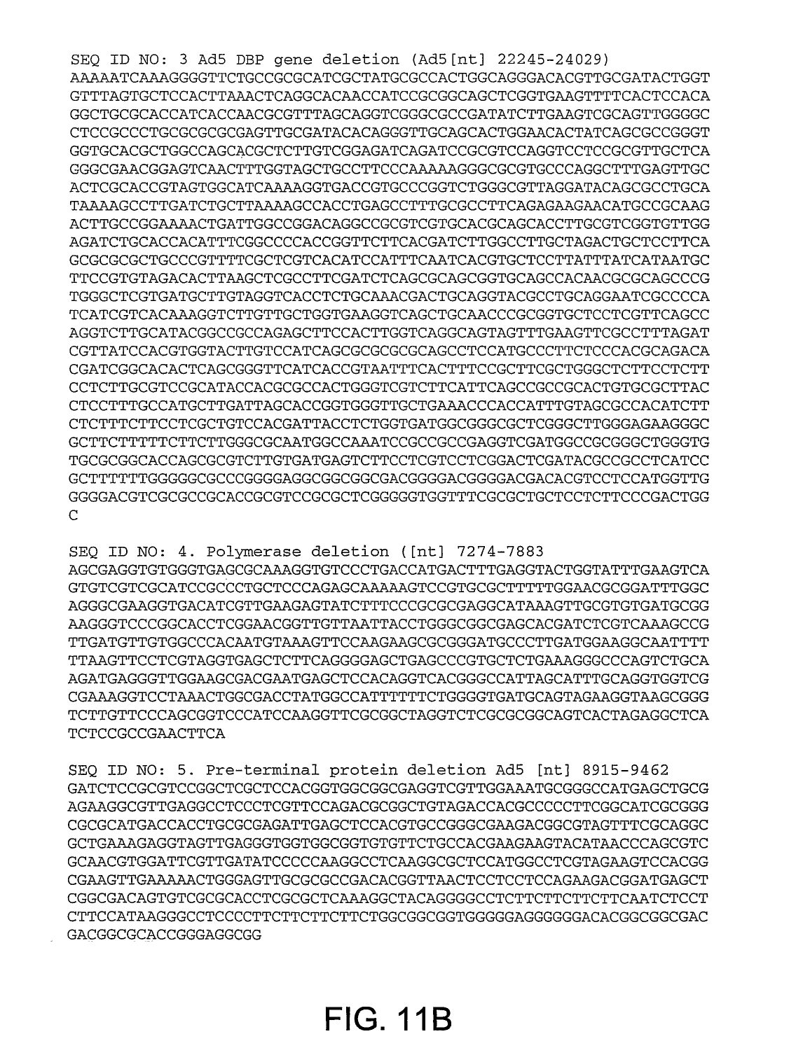

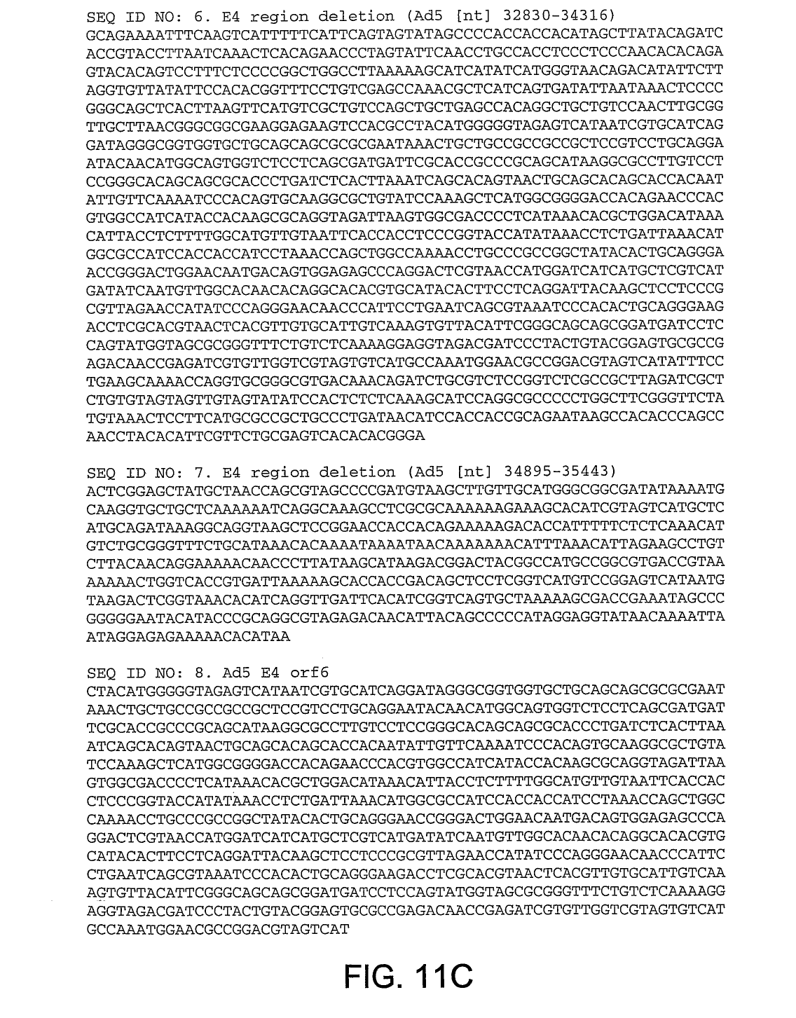

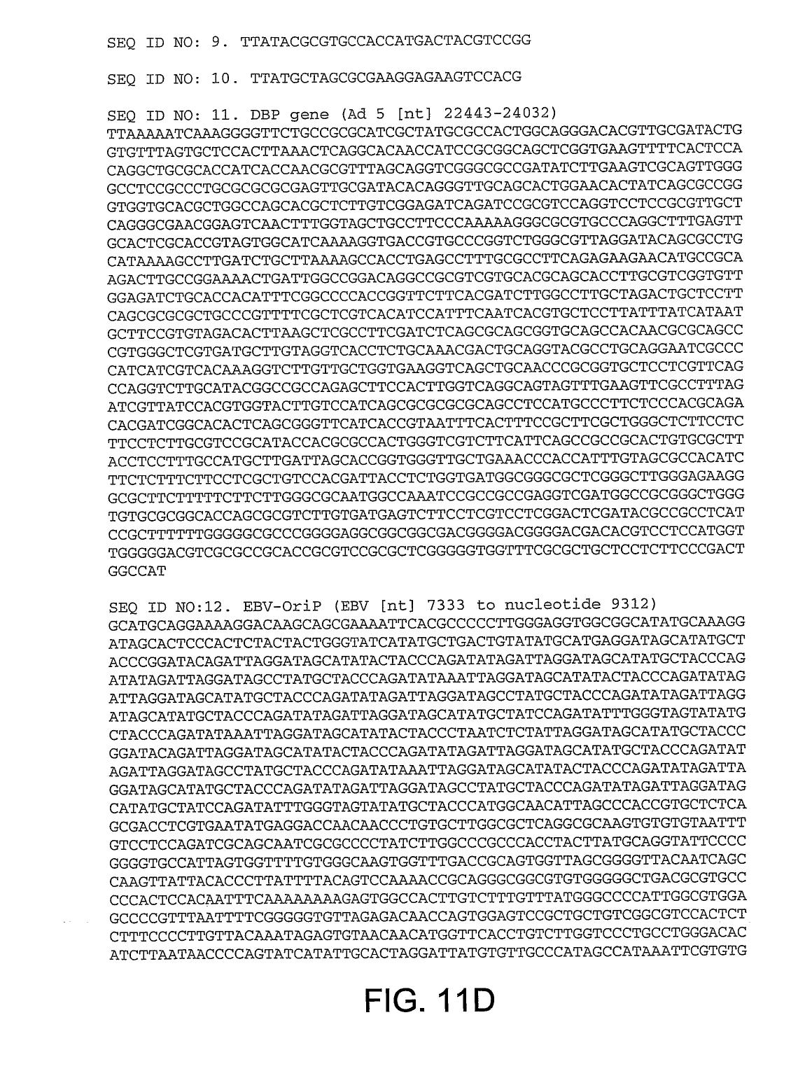

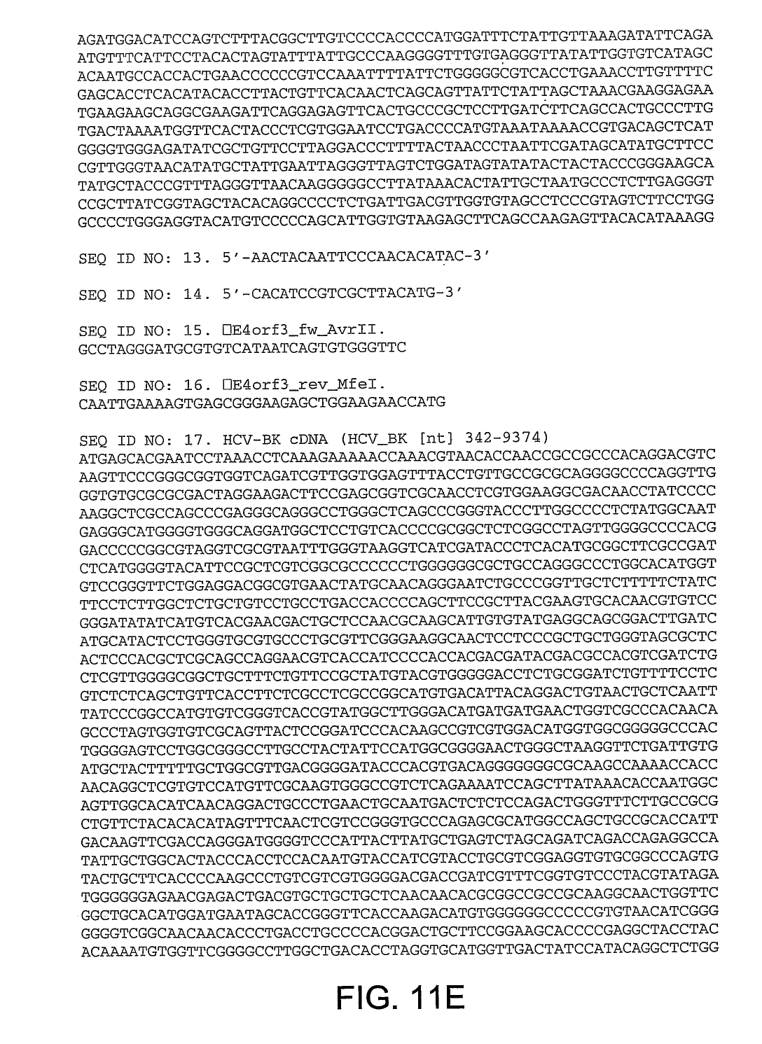

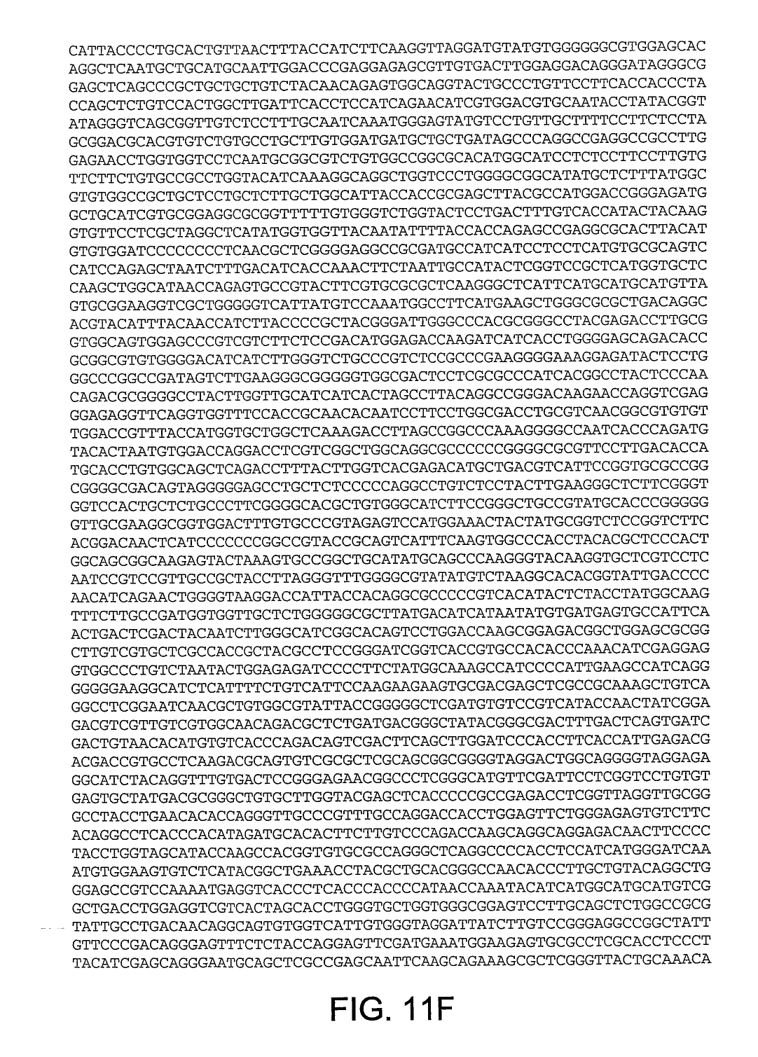

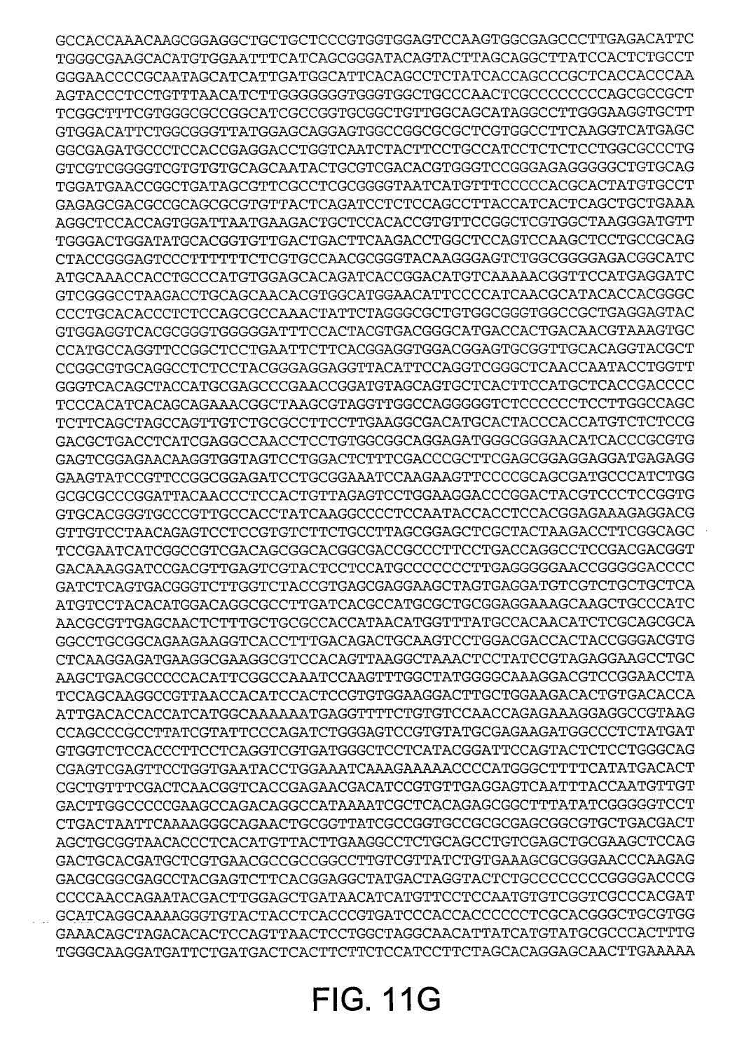

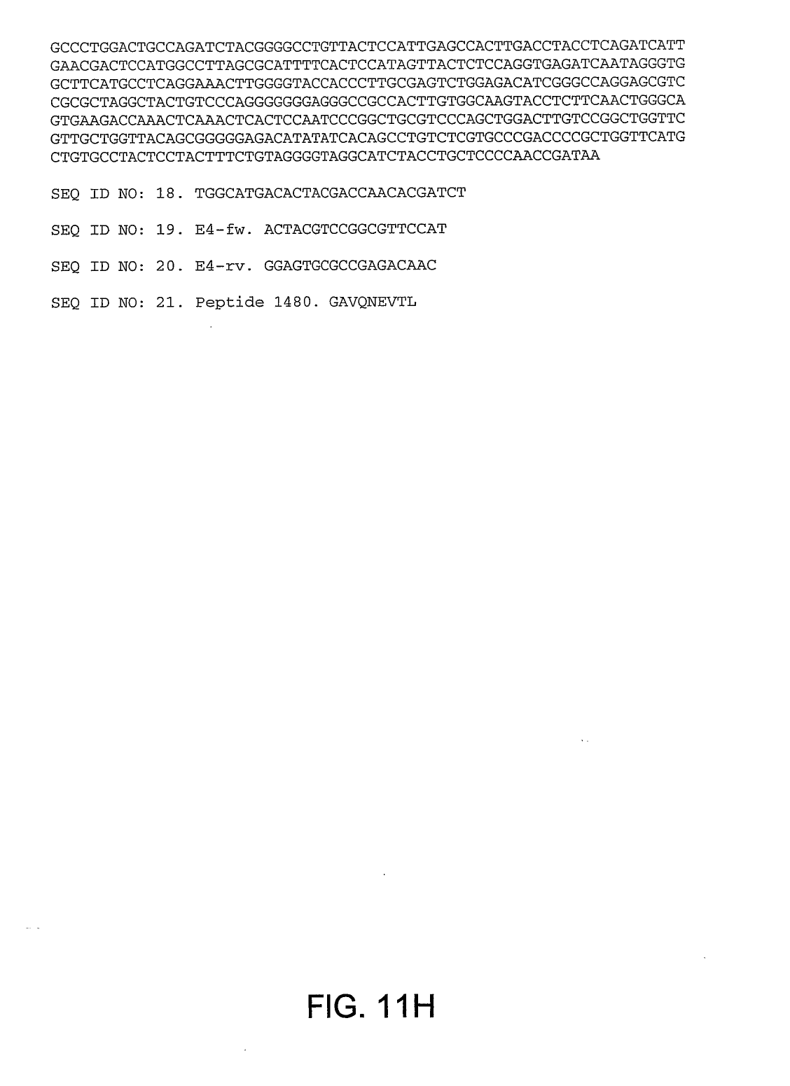

Sequencing may be carried out with commercially available automated sequencers utilizing labeled primers or terminators, or using sequencing gel-based methods. Sequence analysis is also carried out by methods based on ligation of oligonucleotide sequences which anneal immediately adjacent to each other on a target DNA or RNA molecule (Wu and Wallace, Genomics 4: 560-569 (1989); Landren et al., Proc. Natl. Acad. Sci. 87: 8923-8927 (1990); Barany, F., Proc. Natl. Acad. Sci. 88: 189-193 (1991)). FIG. 11 provides the nucleotide and/or amino acid sequences of the polynucleotide and polypeptide sequences (i.e., SEQ ID NOS.: 1-21) described in this disclosure.

Wildtype adenovirus serotype 5 is used as the basis for the specific basepair numbers provided throughout the disclosure. The wildtype adenovirus serotype 5 sequence is known and described in the art; see, Chroboczek et al., 1992 J. Virology 186:280, which is hereby incorporated by reference. One of skill in the art can readily identify the above regions in other adenovirus serotypes (e.g., serotypes 2, 4, 6, 12, 16, 17, 24, 31, 33, and 42) by sequence homology with the regions defined by basepairs for adenovirus serotype 5. Accordingly, it is to be understood that the following examples using the human adenovirus serotype 5 are not meant to be limiting. One skilled in the art would realize that similar plasmids, viruses and techniques could be utilized with a different human adenovirus serotype, for example Ad2. Similarly, the use of human Ads is not meant to be limiting since similar plasmids, viruses and techniques could be utilized for different non-human adenovirus and in particular for chimpanzee adenovirus.

Plasmid Construction

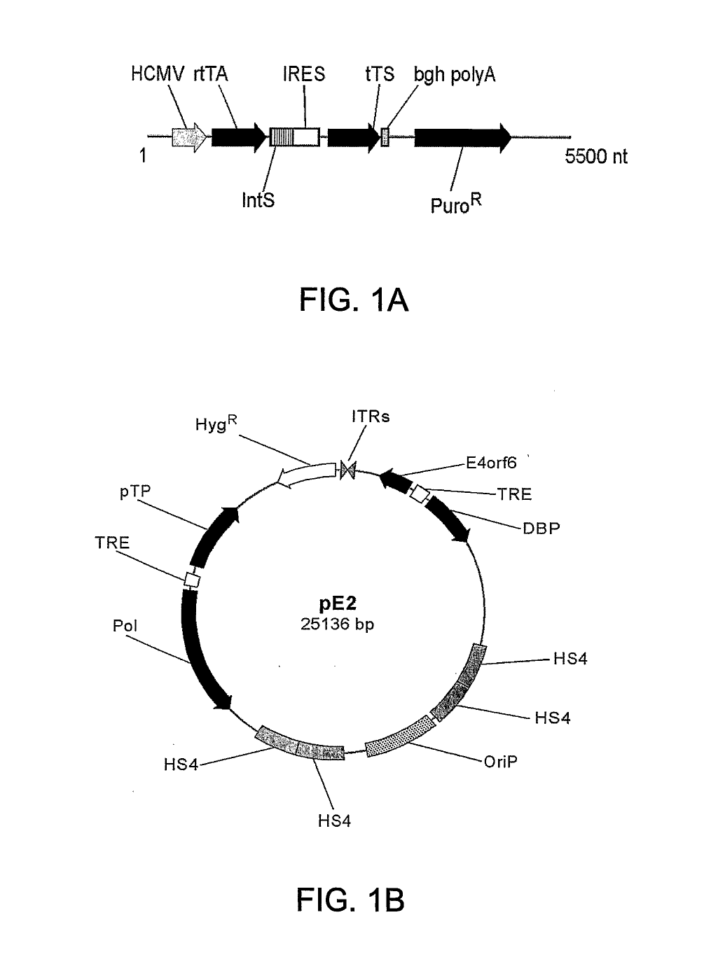

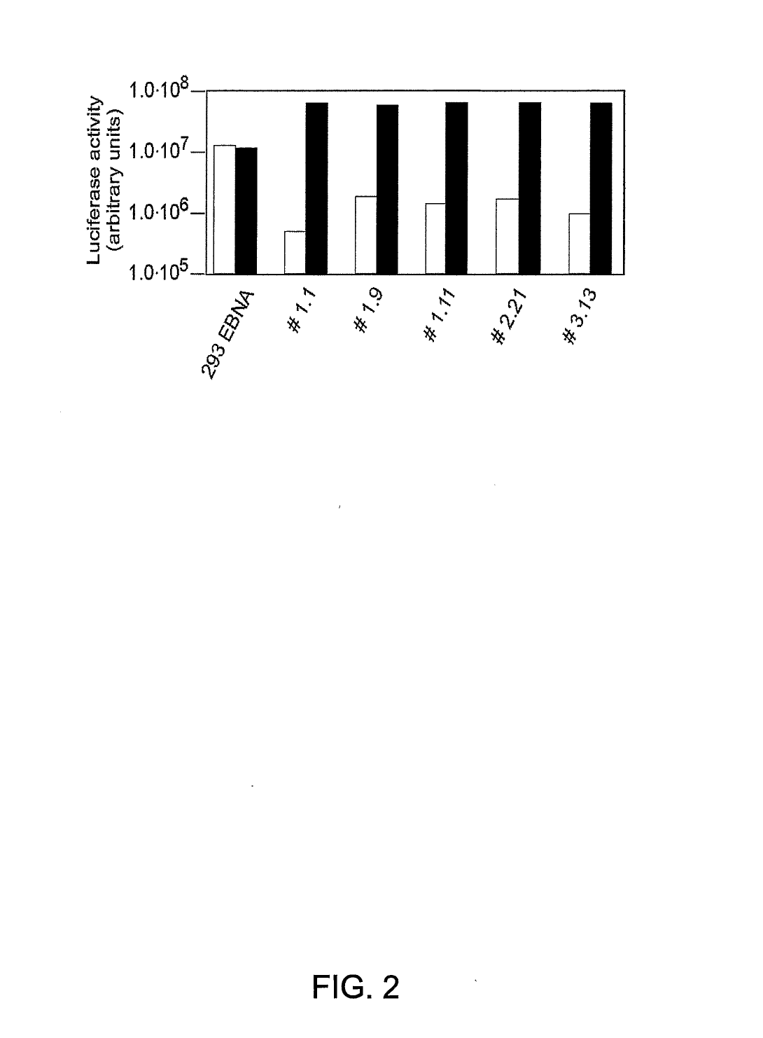

The structure of pIRESTet containing Tet silencer and reverse Tet transactivator expression cassette is described in FIG. 1A. The Tet system was combined in a single expression vector as follows. An EcoRI-ClaI DNA fragment containing the Tet Silencer (tTS) was isolated from the plasmid pUHS6-1 (kindly provided by E. Bujard) and inserted downstream the IRES sequence into the vector pIRES-Neo (Clontech) replacing the Neo gene. The new vector pIRES-tTS was modified with the insertion of rtTA2 gene (from pUHD 52-1, kindly provided by E. Bujard) into the unique EcoRV restriction site downstream the human cytomegalovirus IE promoter, generating pIREStTS/rtTA. FIG. 2 provides a graphic representation of luciferase expression in AdTetLuc infected clones. Briefly, 293EBNA cells and different 293EBNA/Tet clones were infected with AdTetLuc (m.o.i 10) in presence (black columns) or absence (white columns) of 1 □g/ml doxycycline. Luciferase activity in cell lysates was evaluated 48 hours post-infection.

In order to introduce a selection-marker to isolate cell clones stably expressing Tet proteins, a puromycin resistance expression cassette obtained from pPUR vector (Clontech) was inserted in the XhoI site of pIREStTS/rtTA generating pIREStTS/rtTApuro. The structure of pE2 is described in FIG. 1B.

A bicistronic expression vector expressing Ad5 polymerase and pre-terminal protein was constructed by inserting in the vector pBI (Clontech) under the control of the inducible Tet promoter, the ClaI/SphI fragment obtained from plasmid pVacPol including Ad5 polymerase cDNA and the Acc65/EcoRV fragment from pVACpTP containing Ad5-pTP cDNA (pVacPol and pVACpTP were kindly provided by P.C. van der Vliet). A second bidirectional inducible cassette was constructed by inserting into same vector pBI the Ad5 E4 orf6 (Ad 5 [nt] 33193-34077) (SEQ ID NO: 8) obtained by PCR with the oligonucleotides:

(SEQ ID NO: 9)

5′-TTATACGCGTGCCACCATGACTACGTCCGG-3′
and

(SEQ ID NO: 10)

5′-TTATGCTAGCGCGAAGGAGAAGTCCACG-3′

as well as the Ad5 DBP gene (Ad 5 [nt] 22443-24032) (SEQ ID NO: 11) obtained from pFG140 (19).

EBV-OriP (EBV [nt] 7333 to nucleotide 9312; GenBank V01555.) (SEQ ID NO: 12) region derived from pCEP4 flanked by HS4 insulators was obtained by direct cloning into BamHI site of pJC13-1 (9). Ad5 ITR junction was amplified by PCR from pFG140 using the oligonucleotides:

| | |
|---|---|
| 5′-AACTACAATTCCCAACACATAC-3′ | (SEQ ID NO: 13) |
| and | |
| 5′-CACATCCGTCGCTTACATG-3′. | (SEQ ID NO: 14) |

Finally, the tk-hygromycin-B phosphotransferase (HPH) cassette derived from pCEP4 (Invitrogen). All the elements composing pE2 were sequentially transferred into pBI-pol/pTP vector finally generating pE2.

Construction of pAd5ΔE₁₋₄

An Ad5ΔE1-E3 backbone deleted of E2b genes was obtained by transferring the partial deletion of Ad5 polymerase (Ad5 [nt] 7274-7883) (SEQ ID NO: 4) and pre-terminal protein (Ad5 [nt] 8915-9462) (SEQ ID NO: 5) from pAdCMV/LacZ/ΔPol vector (kindly provided by A. Amalfitano(4)) and Ad5dl308Δ_{pTP}β-gal (kindly provided by J. Schaack) (45) respectively into MRKpAd5E3 (52). Additionally, a site specific mutagenesis of the polymerase start codon ATG to CTG was also performed finally obtaining a pAdS ΔE1,E3,E2B vector. pBluescriptKSII+ (Stratagene) that contains the BamHI/XhoI fragment of Ad5 ([nt] 21563-24797) deleted of the DraI-MscI fragment (Ad5 [nt] 22445-24029) comprising DBP gene was kindly provided by Rocco Savino.

***The pAd-ΔE₁₋₂ vector was obtained by homologous recombination co-transforming ΔDBP fragment and AdΔE1,E3,E2B vector into E. Coli Bj5183. Deletion of the complete E4 unit (nt] 32830-34316 and [nt] 34895-35443]) except for orf3 was performed as described below. The orf3 region with AvrII and MfeI restriction sites at termini was amplified by PCR (ΔE4orf3_fw_AvrII: 5′-GCCTAGGGATGCGTGTCATAATCAGTGTGGGTTC-3′ (SEQ ID NO: 15); ΔE4orf3_rev_MfeI: 5′-CAATTGAAAAGTGAGCGGGAAGAGCTGGAAGAACCATG-3′ (SEQ ID NO: 16)) and cloned in an E4-shuttle vector digested with the same enzymes. The E4orf3 maintains E4 promoter and polyA signal. pAd5ΔE₁₋₄orf3⁺ vector was obtained co-transforming such DNA with pAd5ΔE₁₋₂ vector in E. Coli BJ5183.

All expression cassettes were constructed in the context of an Ad5-shuttle vector that contains in addition to CMV promoter and BGH polyA signal for transgene expression, the Ad5 sequences [nt] 1-450 (left) and [nt] 3511-5792 (right) to allow the insertion in the E1 region of pAd5ΔE₁₋₄orf3⁺ by homologous recombination in E. Coli BJ5183 as described (53). EGFP cDNA was obtained from pEGFP plasmid (Clontech, BD Bioscience, San Jose, Calif., USA) then cloned in Ad-shuttle plasmid obtaining pShAd5 EGFP. The HCV-BK cDNA (HCV_BK [nt] 342-9374 (SEQ ID NO: 17)) deleted of 5′ and 3′ Untranslated Terminal Repeats (UTR) was derived from plasmid pCMV(1-9.4) (14).

NS5B ORF was mutated at three amino acid positions corresponding to the catalytic triad of the viral RNA dependent RNA-polymerase (G-2737 to A, D-2738 to A, and D-2739 to G) to abolish enzymatic activity (Nicosia et al., unpublished data). The HCV cDNA fused to an optimized Kozak sequence was cloned in a modified version of pAdS-shuttle obtained by substituting HCMV promoter with MCMV promoter finally constructing pShAd5HCV. Insertion of all expression cassettes in the E1 region of pAd5ΔE₁₋₄orf3⁺ was obtained by homologous recombination in E. coli as described (43).

Cells

293EBNA cell-line (Invitrogen) was cultured in Dulbecco's Modified Eagle's Medium (DMEM) plus 10% fetal bovine serum (FBS), penicillin (100 U/ml), streptomycin (100 μg/ml), 2 mM glutamine and 250 μg/ml G-418 (GIBCO BRL). 293EBNATet cells were selected by using the same medium with 0.5 μg/ml Puromycin. To select 2E2 cells, 90 μg/ml of hygromycin B were added to the previously described medium. Plasmid DNA transfections were performed with Lipofectamine-2000 (Invitrogen) as described by the manufacturer. To obtain a 293EBNA clone expressing reverse Tet transactivator and Tet silencer proteins, one day prior transfection, 1×10⁶293EBNA cells were seeded into 6 cm plates and transfected with 5 μg of a SapI linearized pIREStTS/rtTApuro. 48 hours post-transfection, cells were trypsinized and seeded into 15 cm plates in puromycin containing DMEM. Resistant clones were isolated and subsequently screened with a recombinant Ad5 carrying a Tet-luciferase cassette. 5×10⁵ cells of each clone were seeded in triplicate into 24-well plates and infected with Ad5 Tet-luc with a multiplicity of infection (moi) of 10 with and without doxycycline. 24 hours post-infection cells were harvested and the luciferase activity was measured in cell lysate (luciferase assay system; Promega). Both induction and silencing of gene expression were scored for each clone as a ratio with relative light unit (rlu) values obtained in control experiments made in parental 293EBNA cells.

To obtain 2E2 packaging cell line, 293EBNATet cells were transfected with pE2 vector following the protocol described above. Stable transfectants were selected using DMEM containing 90 μg/ml of hygromycin B. Resistant clones were expanded and screened by transfection of an Ad5ΔE₁₋₂EGFP DNA. Positive clones were identified by CPE appearance and confirmed by serial passaging of the Ad5ΔE₁₋₂EGFP vector. Episome copy number from 1×10⁶ 2E2 cells (n=3) was evaluated by quantitative real-time PCR directly on extra-chromosomal DNA. Probe (5′-FAM-TGGCATGACACTACGACCAACACGATCT-3′-TAMRA) (SEQ ID NO: 18) and primers E4-fw (5′-ACTACGTCCGGCGTTCCAT-3′) (SEQ ID NO: 19) and E4-rw (5′-GGAGTGCGCCGAGACAAC-3′) (SEQ ID NO: 20).

Virus Amplification and Titration

The production of the multiply deleted virus was carried out in 2E2 packaging cell line. Adenovirus genomes were released from the respective plasmids by PacI digestion and transfected in 2E2 cells in presence of 1 μg/ml doxycycline. 4 to 6 days post-transfection, cells were lysed by three freeze/thaw cycles, and ⅕^{th} of the lysate was used to amplify the virus by serial passaging. Large scale amplification was performed by infecting 2E2 cells seeded into two-layer cell factories (NUNC). Adenoviral vectors were purified by CsCl gradients, dialyzed and quantified by real-time PCR. Infectivity of the CsCl purified vector was evaluated on 2E2 as tissue culture infectious dose 50% (TCID₅₀) (43).

Southern Blot Analysis

pE2 replication was evaluated by Southern blot analysis. 293EBNATet cells were seeded in 6-cm dishes and transfected by Lipofectamine-2000 (Invitrogen) with 5 μg of pE2 vector with or without doxycycline (1 μg/ml). Extra-chromosomal DNA was isolated after 48 hours by Hirt method (22). Then, DNA was digested with NotI and DpnI and subjected to Southern analysis according to standard procedures using a ³²P DNA probe. Signals were detected by autoradiography with the Phosphor Imager™ system (Molecular Dynamics).

Episomal DNA from stable pE2 clones was extracted following the Hirt protocol, digested with BamHI and analyzed by Southern blotting using ³²P-labeled pE2 DNA as probe.

Western Blot Analysis

Analysis of protein expression was performed 48 hr post transfection, as follows. 2E2 cells were washed twice with phosphate-buffered saline (PBS) and lysed by adding 0.5 ml of RIPA buffer (1×PBS, 1% NP−40, 0.5% sodium deoxycholate, 0.1% SDS, 0.05 mM PMSF) per 6-cm plate. Plates were incubated 1 hr on ice then soluble proteins were collected from cell lysates after centrifugation at 10,000×g at 4° C. Western blot analysis was performed on 30 μg of proteins. Samples were separated on 10% SDS PAGE and blotted onto Protan nitrocellulose membranes (Schleicher and Schuell). The membranes were incubated with rabbit anti-sera directed against polymerase or pTP and with anti-DBP mab (clone H2-19, kindly provided by F. Graham, Mc Master University, Hamilton, Canada). After incubation with horseradish peroxidase-conjugated secondary antibodies, proteins were detected by Supersignal West Pico chemiluminescent substrate (PIERCE). HCV protein expression was detected by using the following reagents: anti-core monoclonal antibody (mab) B 12.F8 (kindly provided by M. Mondelli, University of Pavia); anti-E2, mab 185.C7; anti-NS3 mab 10E5/24; anti-NS5a, rabbit polyclonal antiserum; anti-NS5b mab 20B6/13.

Immunization Protocol and Analysis of the Immune Response

6 to 8-week-old female C57BL/6, A2.1 and CB6F1 mice (Charles River Breeding Laboratories) were immunized by injecting the virus into both quadriceps. The immune response was analyzed 3 weeks post-injection.

Rhesus macaques were immunized by injecting the virus into the quadriceps muscle. The immune response was analyzed at weeks (W) 4, 6, 8, and 12 post-injection.

Antibody titers against E2 protein was determined by ELISA as described by Zucchelli, S. et al. (55). Cellular immune-response was evaluated as described below. Pools of 15mer overlapping peptides encompassing the entire sequence of HCV Core, NS3, NS4, NS5a and NS5b proteins were used to reveal HCV-specific IFN-γ-secreting cells. In some experiments a 9-mer peptide containing a CD8+ epitope was used to evaluate the immunoresponse (pep1480, GAVQNEVTL (SEQ ID NO: 21) from HCV NS3 protein). IFNγ-secreting cells were quantified by IFNγ enzyme-linked immunospot assay (ELIspot) as follows. Multiscreen 96-well filtration plates (Millipore) were coated with 100 μl of anti-mouse IFNγ Mab (PharMingen), incubated overnight a 4° C., then washed with 1×PBS and blocked 2 h with 200 μl of R10 medium per well. Splenocytes were prepared from immunized mice and resuspended in R10 medium (RPMI 1640 supplemented with 10% fetal calf serum, 2 mM L-glutamine, 50 U of penicillin per ml, 50 μg of streptomycin per ml, 10 mM HEPES, 50 μM 2-mercaptoethanol) then plated on Multiscreen 96-well plates coated with anti-IFNγ mab, at density of 2.5×10⁵ or 5×10⁵/well. Splenocytes were then incubated for 24 h in presence of 200 ng/well of peptide pools. After extensive washing (1×PBS, 0.005% Tween), biotinylated rat anti-mouse IFN-γ antibody (PharMingen, San Diego, Calif.) was added and incubated 3h at room temperature. Finally streptavidin-alkaline phosphatase (PharMigen) and 1-Step NBT-BCIP Development Solution (Pierce, Rockford, Ill.) were added to the well. Spots were counted by using an ELIspot reader (Bioline).

IFN-γ intracellular staining and FACS analysis was performed as follows. Splenocytes prepared as described for Elispot assay, were incubated overnight with peptide pools in R10 medium containing brefeldin (GolgiPlug, PharMingen), which inhibits protein transport. Cell blocking was performed by incubating cells in FACS buffer (PBS w/o Ca and Mg, 1% FCS, 0.01% NaN₃) with saturating amount of purified anti-mouse CD16/CD32. After wash with FACS buffer, APC-conjugated anti-mouse CD3e, PE-conjugated anti-mouse CD4 and PerCP-conjugated anti-mouse CD8a antibodies were added to the cells and incubated at room temperature for 30 min. Cells were then permeabilized at 4° C. for 20 min using Cytofix/Cytoperm Plus (with GolgiPlug) Kit. After a wash with Perm/Wash, FITC conjugated anti-mouse IFN-γ was added and the cells were incubated at room temperature for 30 min. After the final staining step, the cells were washed and fixed in 1% formaldehyde. Data were acquired using a FACSCalibur (Beckton & Dickinson) and analyzed using Cell-Quest software (BD Biosciences). All antibodies and secondary reagents were purchased from BD PharMingen (San Diego, Calif.).

Monkey PBMC were isolated from EDTA-treated peripheral blood by Accuspin Istopaque tubes (Sigma Aldrich cat A0561) according the manufacturer's instructions. Briefly, blood was transferred to the Accuspin tubes containing an equal volume of HBSS (Hank's Balanced solution Gibco cat 14175-053) and centrifuged at 800 g for 15 min at RT. The PBMC band was collected and washed 1X with B13SS, 1X with R10 and finally resuspended in R10. g-IFN-Elispot was run as described above, the only difference being in the amount of cells plated in each well (2×10⁵ and 4×10⁵/well).

Vaccinia Virus Challenge

Immunized mice were injected i.p with 5×10⁶pfu of the VV-NS. Paired ovaries from individual mice harvested 5 days later were homogenized, freeze-thawed three times and titrated by plating 10 fold dilutions on a monolayer of Hu143TK⁻ cells. Titers were read 48 hrs later by staining with 0.5% crystal violet.

### EXAMPLES

Examples are provided below to further illustrate different features of the present invention. The examples also illustrate useful methodology for practicing the claimed invention. The examples are not intended to limit the invention.

### Example 1

### Development of a Cell Line Co-expressing the Tet-S/rtTA2

Stable clones obtained by pIREStTS/rtTApuro transfection in 293EBNA cell lines followed by puromycin selection (see Material and Methods), were screened by using a first generation Ad vector carrying a Tet inducible luciferase expression cassette (Ad Tet-luc). Puromycin resistant clones were seeded in triplicate into 24-well plates and cells were infected with Ad Tet-luc by using a moi of 10 and maintained with/-out 1 μg/ml of doxycycline. The luciferase expression was measured 24 hours post-infection in the crude cell lysate. Clones showing an induction of luciferase activity over 20-fold were selected and expanded. As it is shown in FIG. 2, the co-expression of Tet silencer reduced the basal level of luciferase in all selected clones. Clone 1.1 (named 293EBNATet) showing 25-fold reduction to the basal expression and associated with the best induction ratio (more than 100-fold), in presence of doxycycline, was selected and expanded.

### Example 2

### Construction of the E2/E4 orf6 Adenoviral Amplicon (pE2)

To functionally complement an Ad vector deleted of all early genes, we constructed an Ad5-based amplicon containing the following elements: i) the latent origin of replication of EBV (Ori-P) for stable maintenance in the nucleus of dividing cells expressing the EBNA-1 protein (48); ii) the tk-hygromycin B selection marker; iii) an Ad5 viral ITRs junction derived from pFG 140 (19) to allow plasmid replication in an Ad-based fashion; iv) the Ad5 E2 (polymerase, pre-terminal protein and DNA binding protein) and E4-orf6 genes arranged in two divergent transcriptional units under the control of bi-directional tetracycline-inducible promoters. Two chicken β-globin HS4 insulator dimers (9) flanking the OriP element were also introduced to reduce the enhancer effect of the OriP on the E2 and E4 orf6 distal promoters (17). The structure of the resulting plasmid (pE2) is shown in FIG. 1B.

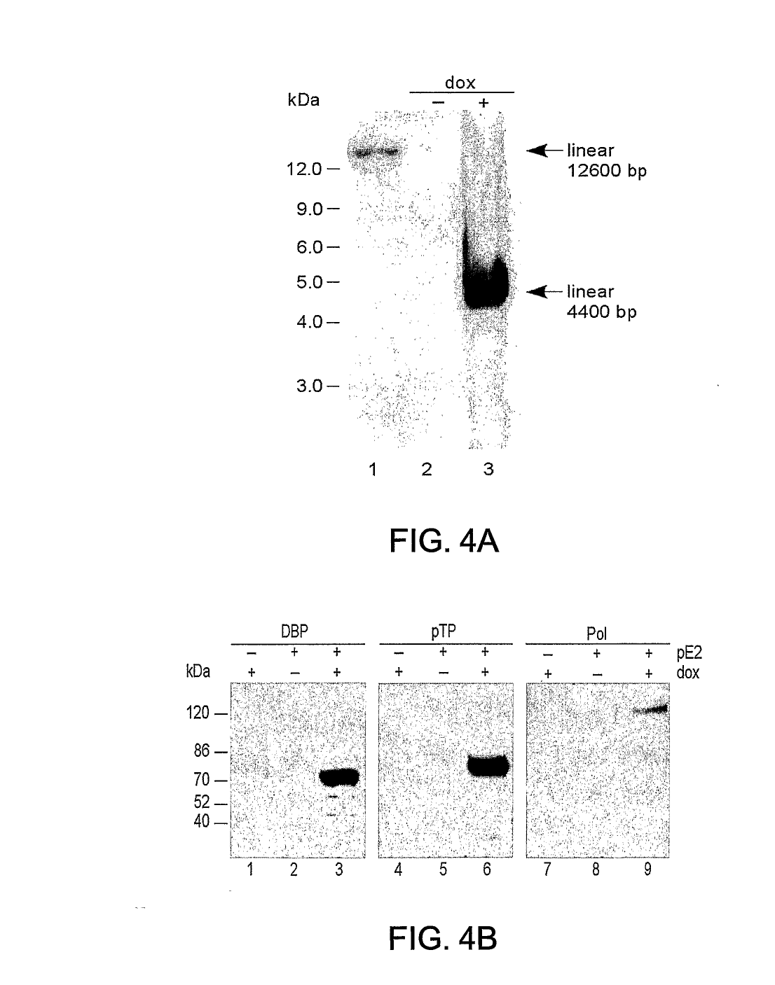

Induction of E2 gene expression upon addition of doxycycline in the medium of pE2 transfected 293EBNATet cells was measured by Western blot. As shown in FIG. 4B (lanes 2, 5 and 8), no protein expression was detected 48 hours post-transfection in the absence of effector drug, while strong expression of all E2 proteins was evident when transcription was induced by adding 1 μg/ml of doxycycline (lanes 3, 6 and 9). Similar results were obtained at 4 and 6 days after transfection: (data not shown).

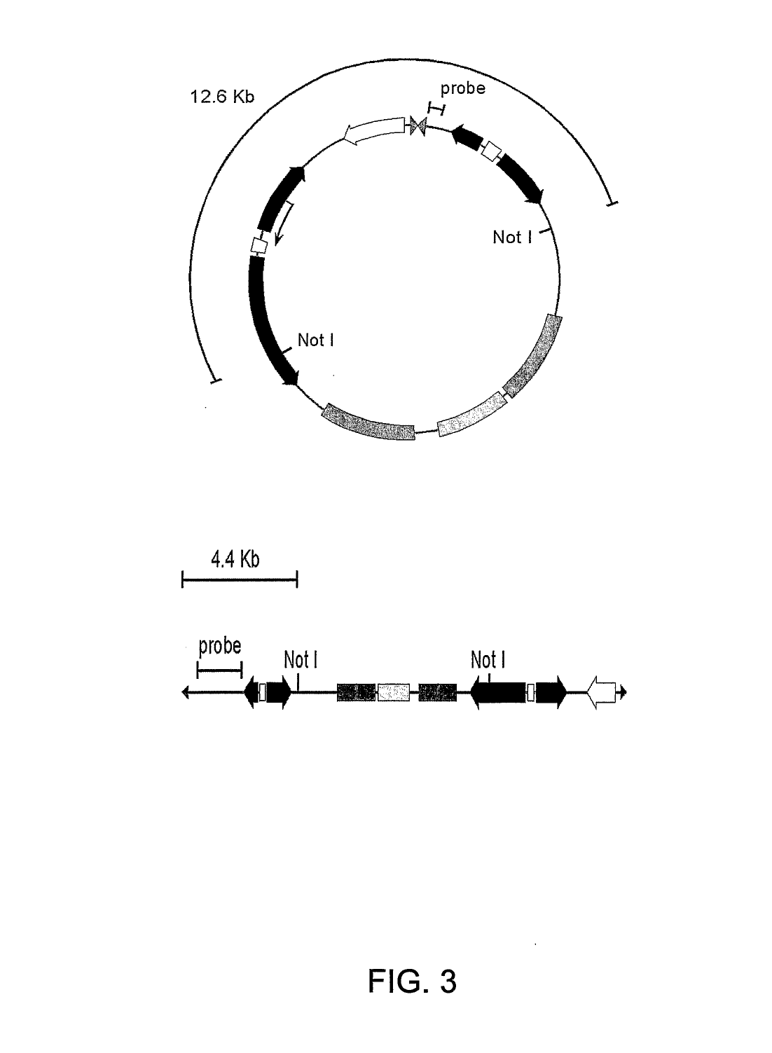

Since both cis- and trans-acting elements necessary for Ad replication are present in the above described system, we tested whether induction of E2 gene expression would also trigger pE2 DNA replication in 293EBNATet transfected cells. Plasmid replication was detected by Southern blot 48 hours post-transfection on total DNA. Samples were digested first with DpnI to get rid of the input plasmid DNA and then with NotI to differentiate between native circular plasmid form and linear forms replicated via Ad ITRs (FIG. 3). Blots were hybridized to a DNA probe derived from pE2 as indicated in FIG. 3, and showed a plasmid-derived band of 12.6 Kb for the circular form of pE2 (FIG. 4A, lane 1). When total DNA from pE2 transfected cells was analyzed, a band of the expected size for the replicated amplicon was visible only when cells were induced with doxycycline (FIG. 4A, lane 2 and 3). No evidence of DNA replication was detected from transfection of a pE2 plasmid derivative deleted of ITRs junction (data not shown). These data indicated that the tTS/rtTA silencing/activation system allows the complete shut-off of pE2 functions even when the plasmid is present in high copy number after transient transfection, but can support efficient replication of the amplicon in an Adenovirus-specific fashion upon doxycyclin induction of early gene expression. They also provided solid evidence in favor of the pE2 amplicon in combination with 293EBNATet cells as a suitable system for rescue and growth of Adenovirus vectors deleted of the early genes.

### Example 3

### Generation of E1, E2, E4 Complementing Cell Line

It was observed that induction of E2 gene expression resulted in replication of pE2 as a linear DNA through activation of the adenovirus replication machinery. pE2 was used to transform 293EBNA cells expressing the Tet transcriptional silencer (tTS) and the reverse Tet transactivator 2 (rtTA2), thus obtaining the 2E2 stable cell line. 2E2 cells produced higher levels of polymerase, precursor terminal protein (pTP) and DNA binding protein (DBP) than 293 cells infected with Ad5 first generation (FG) vector when doxycycline was added to the medium. When induced, the expression of E2 and E40RF6 genes efficiently supported the amplification of a multiply deleted Ad5 vector that lacks E1, E2, E3 and E4 genes to a level comparable to a first generation (FG) adenoviral vector.

Briefly, 293EBNATet cells transfected with pE2 were selected in presence of hygromycin B as described in Material and Methods. Individual clones were expanded and screened by looking at rescue and propagation of an Ad5 vector carrying E2 genes deletion. Cells seeded in six-well plates were transfected with the Ad5ΔE₁₋₂ vector in presence of doxy. Seven days post-transfection, cells were lysed by freeze-thaw and 500 μl of cell lysate was used to infect a fresh monolayer of each corresponding clone. Scoring of positive clones was performed by direct observation of CPE at passage 1. The vector was then serially passaged in the selected clones and the propagation was evaluated by real time PCR. After two serial passages, viral genome reached nearly a plateau of about 1×10¹⁰ genomes per ml of cell lysate that was then maintained even increasing the moi of infection (data not shown). Several clones were identified that contained pE2 as an unarranged intact episome element by evaluating extra-chromosomal DNA with Southern blot analysis.

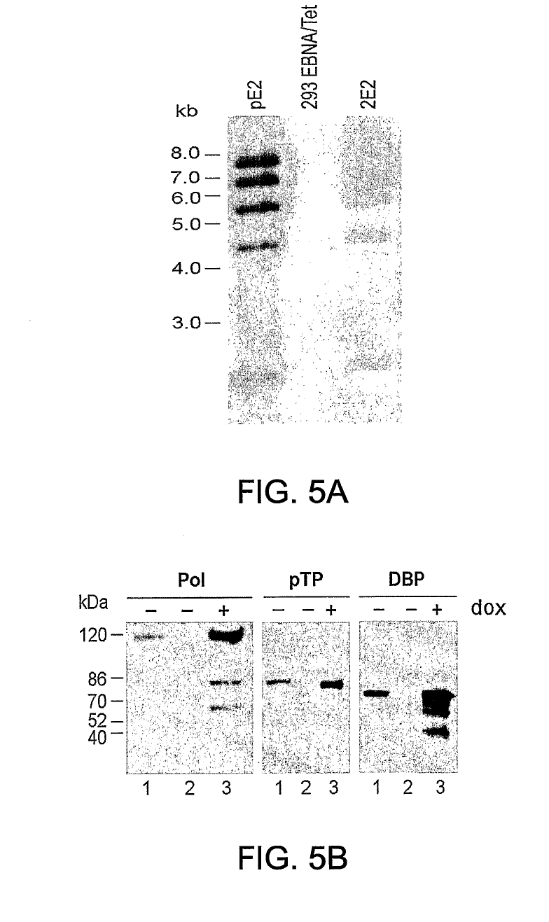

Clone 11, named 2E2, was chosen for the subsequent steps of cell line characterization and ΔE1,E2,E3,E4 vector amplification. Initially, the copy number of pE2 in 2E2 cells were determined (n=3) as an average of 16 copies/cell by real-time PCR on extra-chromosomal DNA. To evaluate the stability of pE2 episomal plasmid in the 2E2 cells over passages, the episomal DNA was extracted after 15 passages following the Hirt method (22) then digested with BamHI and analyzed by Southern blot using the entire plasmid as probe. In FIG. 5A the restriction pattern of episome extracted from the cell line in comparison with the original plasmid is shown. The patterns were identical demonstrating that the episome is stably maintained in the cell nucleus over time and that no rearrangements in the plasmid structure occurred. The stability of the episomal DNA was also confirmed by analyzing the expression of E2 proteins by western blot after 15 passages of the cell line that was similar to what already observed in transient transfection experiments with no detectable expression in absence on doxycycline (FIG. 5B). Cell extracts obtained from non induced 2E2 cells infected with a FG virus was included to compare the E2 protein expression level.

### Example 4

### Construction of a ΔE₁₋₄orf3⁺ Ad5 Backbone

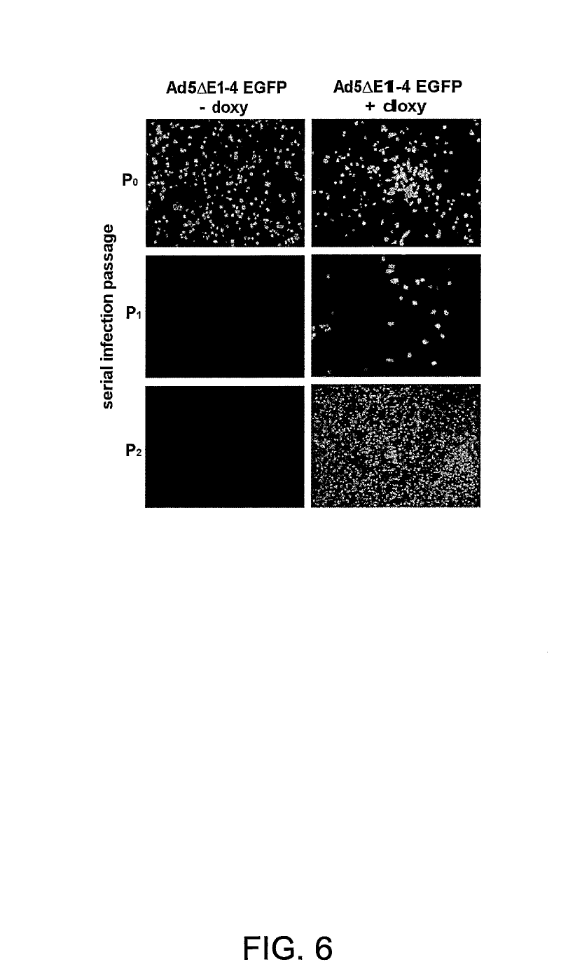

A shuttle vector containing the left ITR and the packaging signal (Ad5 [nt] 1-450) (SEQ ID NO: 1) as well as an Ad5 fragment comprising pIX gene (Ad5 [nt] 3511-5792) (SEQ ID NO: 2) flanking the expression cassette was constructed in order to facilitate the vector construction. Expression cassettes were recombined into Ad5 E1 region by homologous recombination in E. Coli strain BJ5183. To evaluate the efficiency of the new vector system a pAd5ΔE₁₋₄orf3⁺ EGFP was constructed. The pAd5ΔE₁₋₄orf3⁺EGFP vector was linearized with PacI to release the infectious viral DNA from plasmid sequences and transfected into 2E2 cells incubated with or without doxycycline. The results obtained after two serial passages are shown in FIG. 6. EGFP transducing viral particles as well as CPE were produced only when E2 and orf6 genes were induced by doxycycline addition to the medium. orf3 and orf6 proteins co-expression contribute to high titer amplification of Ad vectors deleted of E4 unit (25). No viral particles were generated in absence of complementing gene induction. The vector production plateau was observed just after two serial passages when 100% of cells were EGFP positive (FIG. 6). To evaluate the efficiency of the new cell line in supporting the propagation of the multiply deleted vector to high titer, a large scale preparation was attempted by infecting about 5×10⁸ 2E2 cells seeded in 5 two-layer cell factories (Nunc Inc.). The virus was purified by two passages of CsCl gradient and quantified by real time PCR, finally obtaining a titer of 2×10¹² vp/ml with a production of about 5000 vp/cell. A comparison between FG vectors and multiply deleted vectors expressing EGFP and HCV is presented in Table 1.

TABLE 1
Comparison of Ad5 vectors carrying different deletions. ΔE1, E3
vector was propagated in 293 cells. Multiple-deleted vectors were
propagated in 2E2 cell line. Titers are from CsCl purified virus.

| | | Insert size | Yield | Titer |
|---|---|---|---|---|
| Deletions | Transgene | (Kb) | (vp/cell) | (pp/ml) |
| E1, E3 | EGFP | 1.8 | 1.0 10⁴ | 3.0 10¹² |
| E1, E2, E3 | EGFP | 1.8 | 5.9 10³ | 1.9 10¹² |
| E1, E2, E3, E4orf3⁺ | EGFP | 1.8 | 5.0 10³ | 2.0 10¹² |
| E1, E2, E3, E4orf3⁺ | HCV | 10.5 | 4.9 10³ | 6.2 10¹² |

The theoretical space created in the Ad5 backbone by combining the deletion of all early genes is about 12.4 Kb. The large capacity of the new vector system was exploited to insert an expression cassette for the entire HCV polyprotein gene fused to the mouse cytomegalovirus (MCMV) promoter. The HCV polyprotein expression cassette was constructed by eliminating the 5′ and 3′ untranslated region, by inserting an optimal Kozak sequence upstream core ATG and by mutating the catalytic domain of NS5B replicase to eliminate the enzymatic activity (44). In order to increase the efficiency of transgene expression we substituted the human CMV promoter with mouse CMV promoter that was reported to be 4- to 30-fold more potent in FG adenoviral vectors (1).

The Ad5 ΔE₁₋₄ orf3⁺HCV vector was successfully rescued by transfection in 2E2 cells. The E2 gene expression was induced immediately after transfection by adding doxycycline to the culture medium at a final concentration of 1 μg/ml. Ad5ΔE₁₋₄ orf3⁺HCV vector was amplified by serial passaging in 2E2 cells. Viral genome concentration in crude cell lysate was evaluated by real time PCR as described in Materials and Methods. To obtain a large scale preparation, 2.8×10⁹ 2E2 cells were infected with a moi of about 100 genomes/cell using a crude lysate obtained after four serial amplification passages. Cells were harvested 48 hours post-infection when a full CPE was clearly evident. The final yield of purified virus is reported in table 1. We obtained a production of about 5000 particles per cell not different from a ΔE1E3 FG vector expressing EGFP propagated in 293 cells.

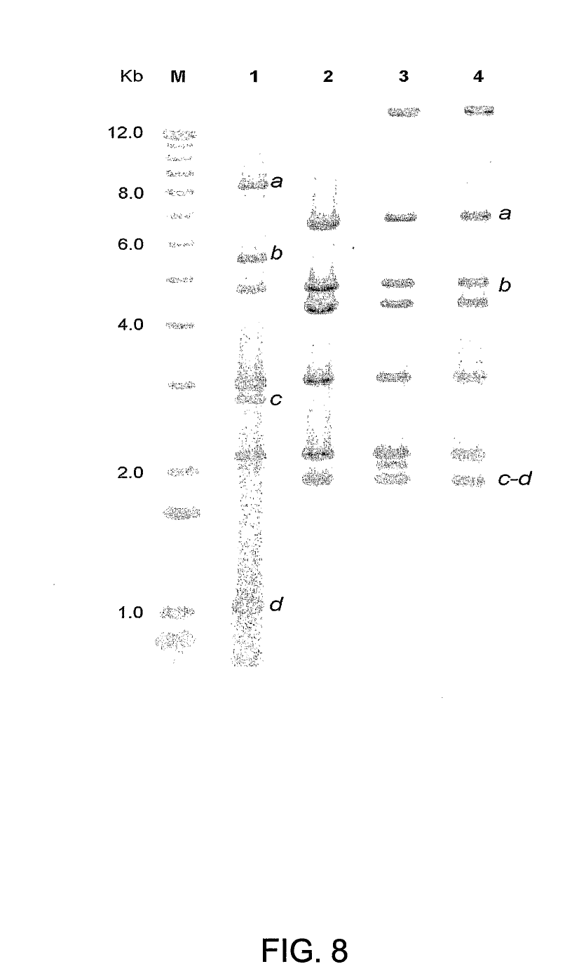

Since the deletions of polymerase and pre-terminal protein involved only a portion of the two genes, the regions of homology between the Ad vector and pE2 episome are theoretically sufficient to rescue the wild-type genes back into the viral genome. To evaluate Ad5ΔE₁₄ orf3+HCV vector structural integrity upon serial passaging as well as to test whether reconstitution of a virus carrying wt early genes could emerge during vector amplification in 2E2 cells, the DNA structure of CsCl purified vector was determined. Comparison of the radio-labeled restriction pattern of the pre-adeno plasmid with the pattern obtained from DNA purified from viral particles after 5 passages is shown in FIG. 8.

A FG plasmid vector was included in the gel (lane 1) to compare the size of the fragments containing the wt genes. The restriction pattern of Ad5ΔE₁₋4orf3+ vector appears to be identical to the parental plasmid and no evidences of emerging vector species carrying rearrangements or wt E2-E4 genes were observed.

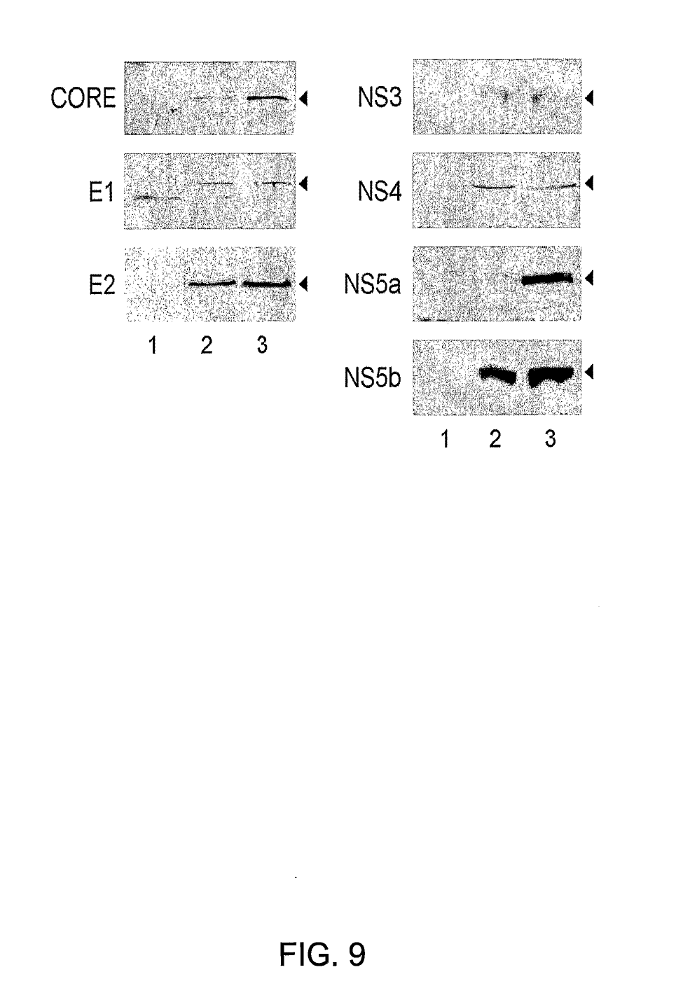

The efficiency of expression of HCV proteins was evaluated by in vitro infection of 293 and HeLa cell lines using different moi of vector. Western blot analysis with specific monoclonal antibodies or polyclonal antisera against HCV core, E1, E2, NS3, NS4, NS5a and NS5B demonstrated the presence of HCV proteins in the infected cells indicating the correct processing of HCV polyprotein (FIG. 9). No unprocessed product was detected.

It should be noted that data indicates that the 2E2 cell line expresses levels of E2a and E2b proteins higher than 293 cells infected by FG vectors. While not wishing to be bound by theory, it is believed that the relatively high levels of E2a and E2b production led to a high yield of multiply deleted vector particles per cell. The yield of multiply deleted particles per cell was consistently comparable to the yield obtained from FG vectors. Moreover, Ad5ΔE₁₋₄orf3⁺ vectors were demonstrated to be stable over serial passaging, in spite of the theoretical possibility that Pol and pTP genes can be rescued in the vector backbone by homologous recombination with pE2. The observed high levels of expression of complementing proteins possibly reduce a selective advantage of an E2b wild type virus over the multiply deleted vector.

### Example 5

### Immunization with Ad ΔE₁₋₄orf3⁺HCV Vector Induces a Strong CMI Response in Mice

Resolution of HCV infection observed in humans and chimpanzees is typically associated with a strong T-cell response directed against multiple epitopes (56, 57). HLA class I-restricted epitopes identified in infected subjects are spread throughout the entire genome without evidences of clustering (reviewed in 58). The efficacy of multiply deleted Ad vector expressing HCV polyprotein to elicit cell medicated immune responses was evaluated in murine immunization experiments. The vector directs the synthesis of the entire polyprotein precursor which is correctly processed into the mature products as demonstrated by western blot analysis of infected cells. Oligopeptides containing HCV-BK CD8+ epitopes that were mapped in different strains of mice were also used to monitor the immunization. CD4+ and CD8+ T cells specific for HCV epitopes were determined by IFN-γ Elispot and intracellular staining (ICS) by using pools of overlapping 15-mer peptides covering the entire sequence of core, E2 and NS proteins.

By immunizing CB6F1 and HLA A2.1 mouse strain, we measured a strong T cell immune response (0.2 to 2.25% of total CD8+ cells) directed against multiple viral determinants. Analysis of splenocytes of immunized mice revealed a CD8⁺-biased immune-response with low levels of CD4+ antigen specific T cells. This characteristic of the immune-response is associated with the modality of vaccination more than with the antigen delivered. Low ratios of CD4+/CD8+ were observed by Casimiro and coworkers in Rhesus immunized with Ad5gag vaccine being the majority of responding cells of the CD8+ phenotype associated with a strong CTL activity (59). On the contrary, genetic immunization with HCV antigens by intramuscular DNA injection led to a more balanced CD4+/CD8+ response in both mice and non-human primates (Nicosia A. unpublished results).

The cellular immunity induced by various amounts of vector was determined by immunizing C57B16 mice with increasing doses of intra-muscularly injected Ad5ΔE₁₋₄ orf3+HCV (from 1×10⁷ up to 1×10¹¹ vp/mouse). Mice were tested 3 weeks post-immunization for T cell response against CD8+ T cell epitope mapped in the helicase domain of NS3 protein (GAVQNEVTL (SEQ ID NO: 21) aa 1629 to 1637 HCV 1b). Freshly isolated splenocytes were incubated overnight with the 9-mer peptide then analyzed by an IFN-γ ELISPOT assay.

As shown in Table 2, the magnitude of the induced T-cell response was dependent on the dose of Ad5ΔE₁₋₄ HCV, with the first positive result observed at 1×10⁸vp/dose. At this dose of vector, 4 mice out 5 developed an immune-response against NS3 with frequency of specific T cell ranging from 100 to 180 CD8+ cells per 1×10⁶ splenocytes.

The data in table 2 summarizes the number of IFNγ spot forming cells (SFC) per million splenocytes obtained from 5 immunized mice. Splenocytes were incubated with the 1480 nonamer (GAVQNEVTL) (SEQ ID NO: 21) that contains a CD8+ epitope mapped in the BK NS3 helicase domain in C57B16 mice. Values obtained from single animal as well as the geomean calculated for each group of immunized mice are reported in the table. The frequency of antigen-specific CD8+ T cells increased according with the dose up a geomean value of 568 with a range of 400-1000 of SFC per million of splenocytes by injecting 10¹¹ vp per animal.

TABLE 2
T cell immune response induced by the Ad5ΔE₁₋4 orf3⁺HCV virus
in C57B16 mice at doses ranging from 10¹¹ down to 10⁷ viral particles.

| | 10⁷ | 10⁸ | 10⁹ | 10¹⁰ | 10¹¹ |
|---|---|---|---|---|---|
| # 1 | 4 | 100 | 200 | 520 | ND |
| # 2 | 5 | 1 | 142 | 280 | ND |
| # 3 | 10 | 178 | 110 | 282 | 356 |
| # 4 | 1 | 176 | 351 | 356 | 536 |
| # 5 | 3 | 118 | 165 | 287 | 962 |
| geomean | 3 | 52 | 178 | 335 | 568 |
| DMSO | 2 | 1 | 1 | 2 | 4 |

### Example 6

### Induction of a Polyspecific CMI Response in Transgenic Mice Expressing Human HLA-A2.1

It is likely that a protective HCV vaccine will need to induce a broad cellular immune response in the general population due to the genetic diversity of human MHC alleles and of the virus. Immunization of mice with the Ad5ΔE₁₋₄ HCV vector induced a strong CD8+ T cell response directed against multiple epitopes of HCV polyproteins. More specifically, the ability of Ad5ΔE₁₋₄orf3⁺HCV vector to elicit cell-mediated immune response directed against multiple HCV epitopes was determined. Due to the restriction of the immune response, HCV specific T cell response elicited by vector immunization was determined in CB6F1 and HLA A2.1 transgenic mice.

Groups of five animals were injected in the quadriceps with 1×10¹⁰ viral particles of Ad5ΔE₁₋₄orf3⁺HCV. Mice were analyzed 3 weeks post-injection by evaluating the strength as well as the quality of the vaccine-induced anti-HCV immunity by an ICS method. Antigen-specific IFNγ secretion from splenocytes was stimulated with seven peptide pools composed by 15-mers overlapped by 10 residues covering core (aa 1 to 190) and the non structural region from NS3 to NS5b proteins (aa 1026 to 3009). Analysis of splenocytes was conducted on pools of 5 mice.

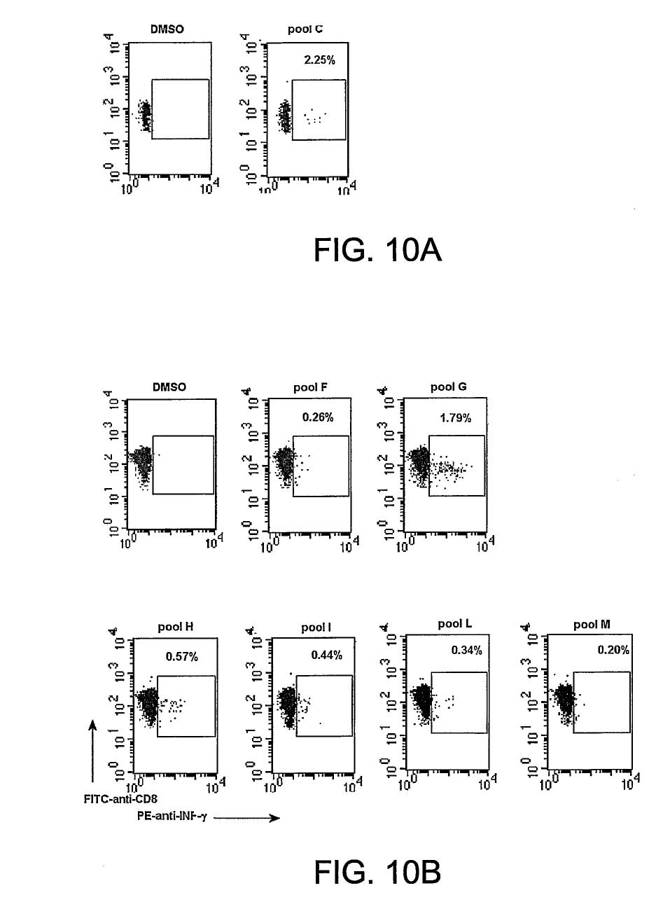

The results shown in FIG. 10 demonstrate that the Ad5_{ΔE1-E4}-HCV vector induces a strong and multispecific T-cell response in transgenic mice expressing human HLA-A2.1. More specifically, the immunization produced a T-cell response directed to all of the peptide pools. Both antigen-specific CD4+ and CD8+ T lymphocytes were observed, however the great majority of the responding cells were CD8+. The frequency of antigen-specific CD8+ lymphocytes varies depending on the antigen from 2.2% of CD8+ T cells directed against the core to 0.2% of CD8+ T responding to the C-terminal part of NS5B.

### Example 7

### Ad5_{ΔE1-E4}-HCV Immunization Elicits a CMI Response in A2.1 Mice

Transgenic A.21 mice were immunized at W=0 and W=2 with either Ad5_{ΔE1-E4}-HCV at a dosage of 10¹⁰pp/mouse/injection or with the corresponding Ad5 shuttle vector (pShAd5HCV) in the dosage of 50 ug/mouse/injection. The immune response of purified splenocytes obtained from animals at week four that were: 1) primed and boosted with Ad5_{ΔE1-E4}-HCV and 2)primed with pSh-Ad5-HCV and boosted with Ad5_{ΔE1-E4}-HCV were analyzed by γ-IFN-Elispot and γ-IFN intracellular staining using peptide pools covering the entire HCV polyprotein.

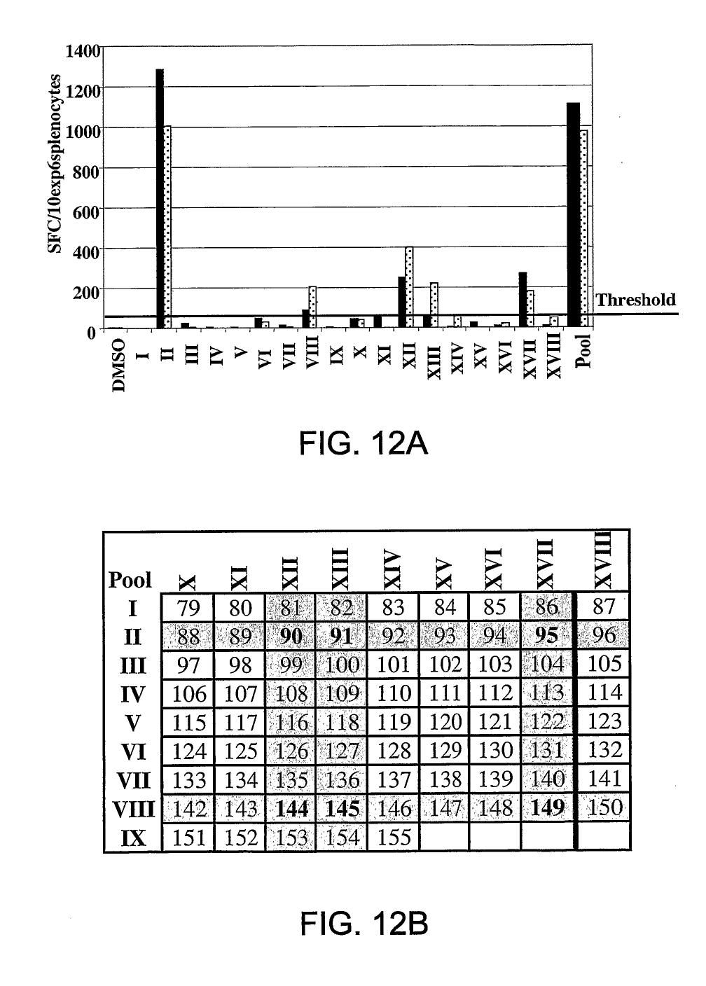

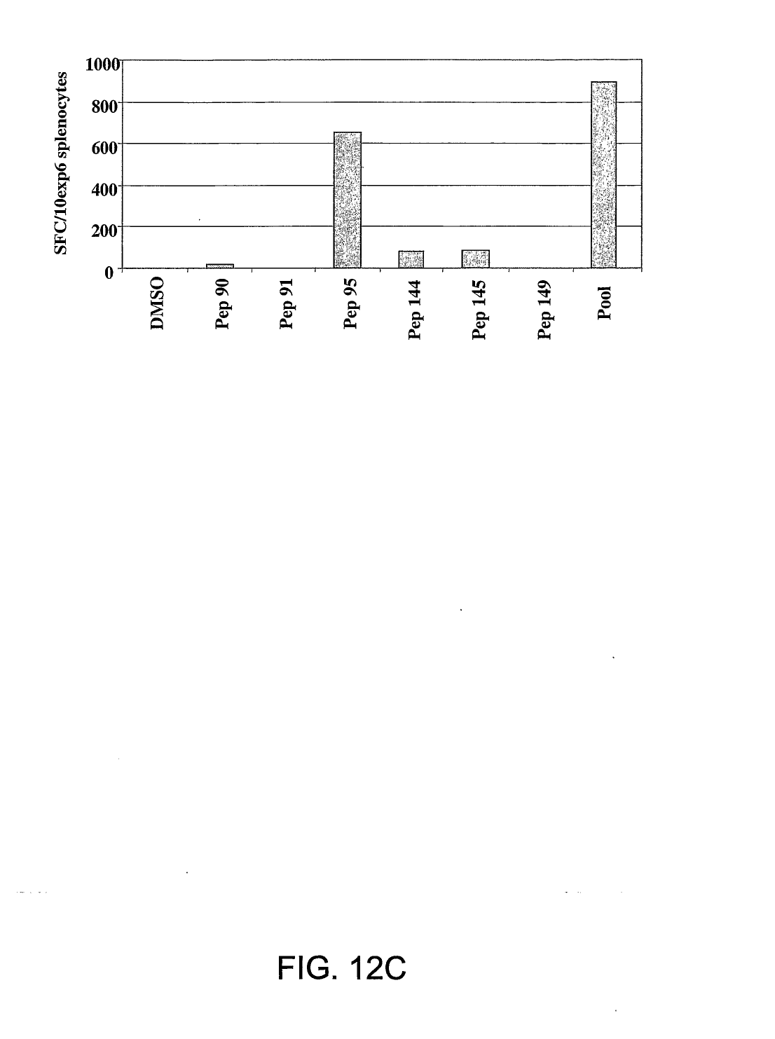

The specificity of the response was determined in an experiment using a sub-set of peptides (from I to XVIII) covering the entire NS3 helicase region. Briefly, splenocytes were isolated from 2 mice primed with Ad5DE1-E4HCV and boosted with pSh-Ad5-HCV. The Elispot results in FIG. 12A indicate that the mice responded with a broad immune response, which is strongly reactive against peptides covering the NS3 region. The data further established that Ad5_{ΔE1-E4}-HCV can be used to both prime and boost an HCV-specific immune response in mice immunized with either Ad5 _{ΔE1-E4}-HCV or pSh-Ad5-HCV.

The points of intersection between pools eliciting a response above the positivity threshold highlight the possible stimulating peptides (FIG. 12B). These peptides were then tested individually in a γ-IFN-Elispot Assay. The results summarized in FIG. 12C demonstrate a specificity/reactivity against peptide 95 (LAAKLSGLGINAVAY) (NS3 aa1403-1417) (SEQ ID NO: 22) epitope. Thus, the immune response elicited in these mice is characterized by a specificity which includes specificity for a CD8+ epitope in a NS3 helicase region previously identified in HCV patients. (69).

### Example 8

### Immunization with Ad5_{ΔE1-E4}-HCV Provides Protection to Challenge with VV-NS

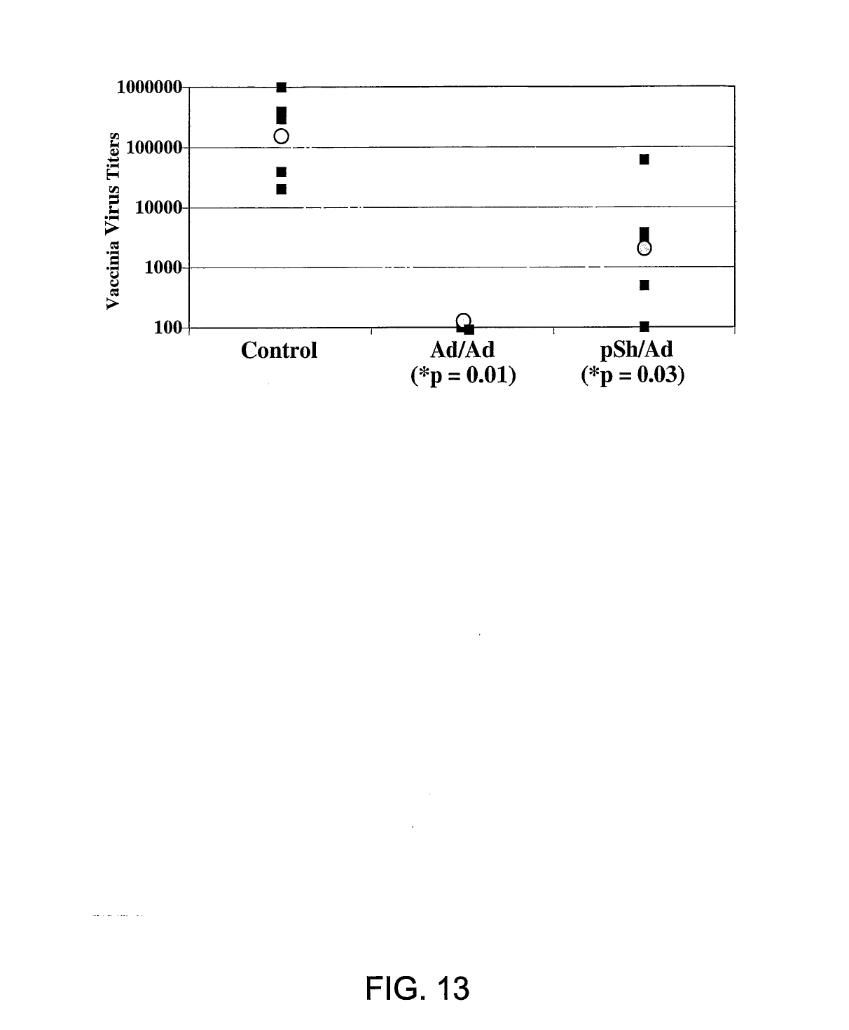

This experiment establishes that the elicited immune response can confer protection against challenge with a recombinant vaccinia virus expressing HCV non structural proteins. In order to determine whether the immune response elicited by immunization with Ad5Δ_{E1-E4} HCV provides protection to a subsequent viral challenge, mice immunized according to the protocol provided in Example 7 were challenged at week 4 with a recombinant vaccine virus expressing HCV non structural region (VV-NS), at a dose of 5×10⁶ pfu. As an experimental control, mice immunized with 2 injections at W=0 and W=2 of Ad5 Δ_{E1-E4}EGFP were used. Paired ovaries were removed five days later and VV was titered. The results presented in FIG. 13 demonstrate a significant decrease in the VV titer of the immunized mice in comparison to the control mice. Grey dots represent geometric mean titres (N=5). *=p<0.05 respect to the control (Mann-Whitney rank)

### Example 9

### Immunization with Ad5_{ΔE1-E4}-HCV Primes an HCV-specific Immune Response in Rhesus Macaques

Rhesus macaques were injected in the quadriceps with 10¹⁰ pp of Ad5_{ΔE1-E4}-HCV. The efficacy of the immunization, was evaluated by γ-IFN-Elispot assay on peripheral blood mononuclear cells (PBMC) at different time points post injection (W=4, W=6, W=8, W=12). The immune response elicited by the injection peaked at week 6 post injection and was directed against multiple HCV epitopes. One of the threes monkeys showed a longevity of response up to 12 weeks post injection. These data indicate that Ad5_{ΔE1-E4}-HCV can be used to elicit immune response in primate animal models.

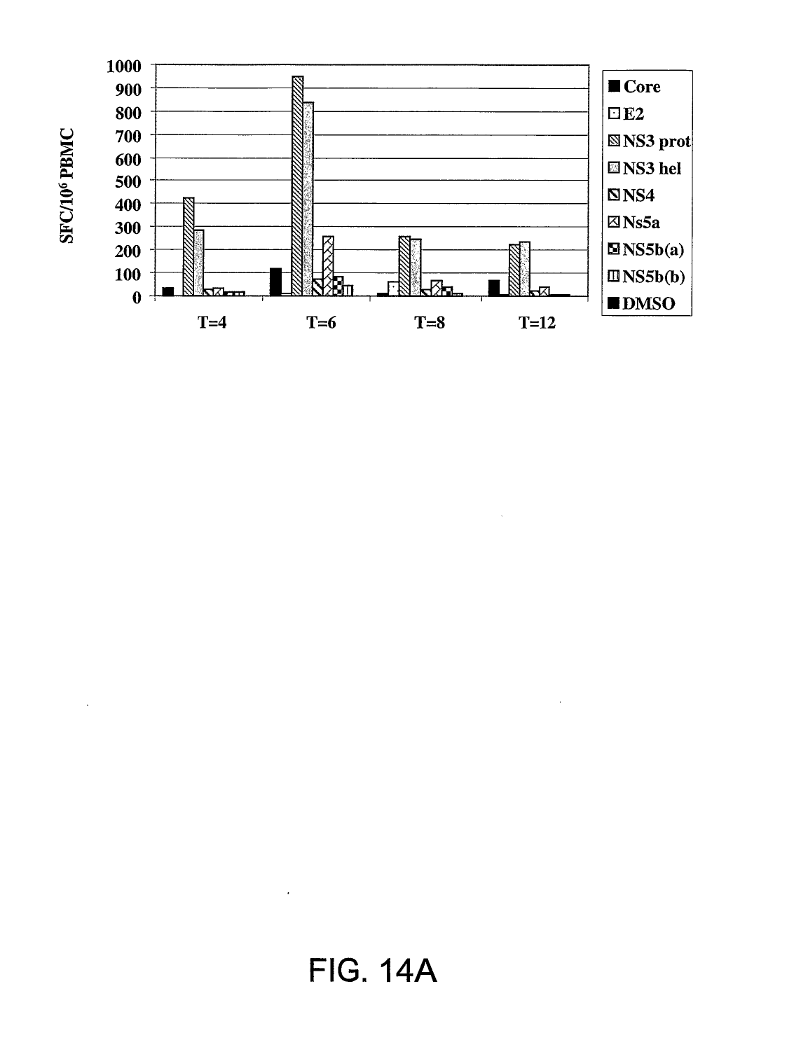

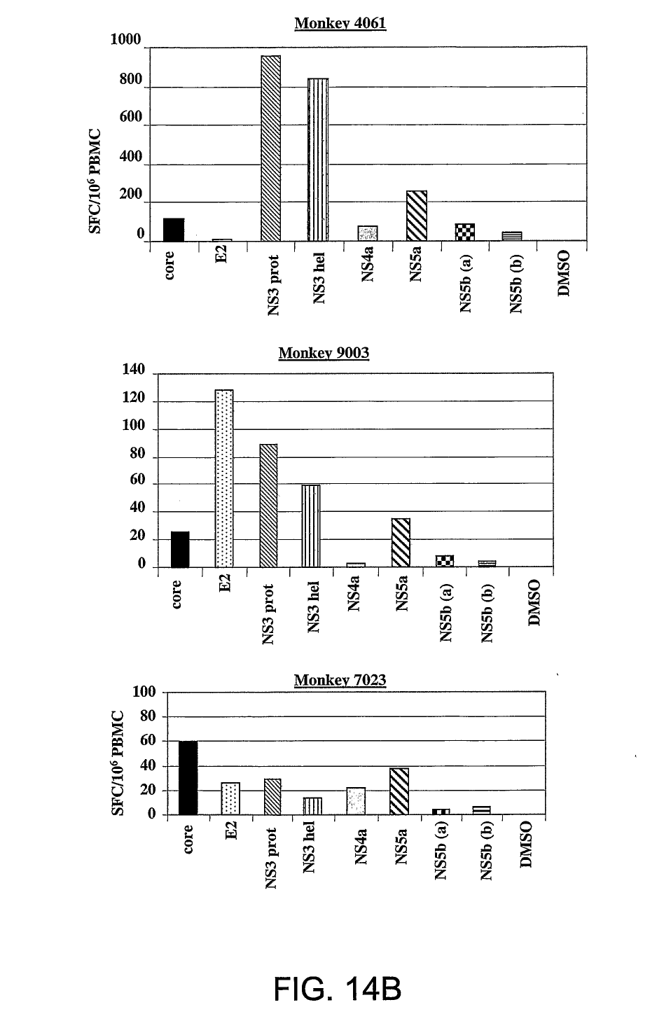

FIG. 14A illustrates the response over the time elicited in monkey 4061 after a single administration of Ad5DE1-E4-HCV. Results are expressed as g-IFN spot forming cells (SFC) per 10⁶ PBMC, at 4, 6, 8, and 12 weeks post-injection. Each bar represents the response to a separate peptide pools.

FIG. 14B illustrates the immune response of three monkeys (Nos: 4061, 9003, 7023)6 weeks post-injection of Ad5DE1-E4-HCV, evaluated in a γ-IFN-Elispot 6 assay. Results are expressed as g-IFN spot forming cells (SFC) per 106 PBMC. Each bar represents the response to a separate peptide pool.

While the present invention has been described with reference to what are considered to be the specific embodiments, it is to be understood that the invention is not limited to such embodiments. To the contrary, the invention is intended to cover various modifications and equivalents included within the spirit and scope of the appended claims.

All references cited throughout the disclosure are hereby expressly incorporated by reference.

### REFERENCES CITED IN THE DISCLOSURE

1. Addison, C. L., Hitt M., Kunsken D. and Graham F. L., Comparison of the human versus murine cytomegalovirus immediate early gene promoters for transgene expression by adenoviral vectors. J. Gen. Virol. 78:1653-1661, 1997.
2. Amalfitano A, Begy C R and Chamberlain J. S., Improved adenovirus packaging cell lines to support the growth of replication-defective gene-delivery vectors. Proc. Natl. Acad. Sci. 93:3352-3356, 1996.
3. Amalfitano A. and Chamberlain J. S., Isolation and characterization of packaging cell lines that coexpress the adenovirus E1, DNA polymerase, and preterminal proteins: implications for gene therapy. Gene Ther. 4:258-263, 1997.
4. Amalfitano A., Hauser M. A., Hu H., Serra D., Begy C. R., Chamberlain J. S., Production and characterization of improved adenovirus vectors with the E1, E2b, and E3 genes deleted. J. Virol. 72:926-933, 1998.
5. Andrews J. L., Kadan M. J., Gorziglia M. I., Kaleko M., Connelly S., Generation and characterization of E1/E2a/E3/E4-deficient adenoviral vectors encoding human factor VIII. Mol Ther 3:329-336, 2001.
6. Bett A. J., Prevec L. and Graham F. L., Packaging capacity and stability of human adenovirus type 5 vectors. J. Virol. 67:5911-5921, 1993.
7. Casimiro D. R., Chen L., Fu T. M., Evans R. K., Caulfield M. J., Davies M. E., Tang A., Chen M., Huang L., Harris V., et al., Comparative Immunogenicity in Rhesus Monkeys of DNA Plasmid, Recombinant Vaccinia Virus, and Replication-Defective Adenovirus Vectors Expressing a Human Immunodeficiency Virus Type 1 gag Gene. J. Virol. 77:6305-6313, 2003.
8. Cheshenko N., Kougliak N., Eisensmith R. C., Krougliak V. A., A novel system for the production of fully deleted adenovirus vectors that does not require helper adenovirus. Gene therapy 8: 846-854,2001.
9. Chung, J. H., Whiteley M. and Felsenfeld G., A 5′ element of the chicken beta-globin domain serves as an insulator in human erythroid cells and protects against position effect in Drosophila. Cell. 74:505-14, 1993.
10. Cooper S., Erickson A. L., Adams E. J., Kansopon J., Weiner A. J., Chien D. Y., Houghton M., Parham P., Walker C. M. Analysis of a successful immune response against hepatitis C virus. Immunity. 10:439-449, 1999.
11. Danthinne, X. and Imperiale, M. J., Production of first generation adenovirus vector: a review. Gene Ther. 7:1707-1714, 2000.
12. Engelhardt J. F., Yang Y., Stratford-Perricaudet L. D., Allen E. D., Kozarsky K., Perricaudet M., Yankaskas J. R., Wilson J. M., Direct gene transfer of human CFTR into human bronchial epithelia of xenograft with E1-deleted adenovirus. Nature Genet. 4:27-34, 1993.
13. Farley, D. C., Brown J. L. and Leppard K. N., Activation of the early-late switch in adenovirus type 5 major late transcription unit expression by L4 gene products. J. Virol. 78:1782-1791, 2004.
14. Fipaldini, C., Bellei B. and La Monica N., Expression of hepatitis C virus cDNA in human hepatoma cell line mediated by a hybrid baculovirus-HCV vector. Virology. 255:302-311, 1999.
15. Fisher K. J., Choi H., Burda J., Chen S. J., Wilson J. M., Recombinant adenovirus deleted of all viral genes for gene therapy of cystic fibrosis.
   Virology. 217:11-22, 1996.
16. Freundlieb, S., Schirra-Muller, C. and Bujard H., A tetracycline controlled activation/repression system with increased potential for gene transfer into mammalian cells. J. Gene. Med. 1:4-12, 1999.
17. Gahn Toni A. and Sugdden B., An EBNA-1-dependent enhancer acts from a distance of 10 kilobase pairs to increase expression of the Epstein-bar virus LMP gene. J. Virol. 69:2633-2636, 1995.
18. Gorziglia M. I., Lapcevich C., Roy S., Kang Q., Kadan M., Wu V., Pechan P., Kaleko M., Generation of an adenovirus vector lacking E1, e2a, E3, and all of E4 except open reading frame 3. J. Virol. 73:6048-6055, 1999.
19. Graham, F. L., Covalently closed circles of human adenovirus DNA are infectious. EMBO J. 3:2917-2922, 1984.
20. Grifman M., Chen N. N., Gao G. P., Cathomen T., Wilson J. M., Weitzman M. D., Overexpression of cyclin A inhibits augmentation of recombinant adeno-associated virus transduction by the adenovirus E4orf6 protein. J. Virol. 73:10010-10019, 1999.
21. Halbert, D. N., Cutt, J. R. and Shenk, T., Adenovirus early region 4 encodes functions required for efficient DNA replication, late gene expression, and host cell shutoff. J. Virol. 56:250-257, 1985.
22. Hirt, B., Selective extraction of polyoma DNA from infected mouse cell cultures. J. Mol. Biol. 26:365-369, 1967.
23. Hodges B. L., Serra D., Hu H., Begy C. A., Chamberlain J. S., Amalfitano A., Multiply deleted [E1, polymerase-, and pTP-] adenovirus persist despite deletion of the preteminal protein. J. of Med. 2:250-259, 2000.
24. Hodges B. L., Evans H. K., Everett R. S., Ding E. Y., Serra D., Amalfitano A., Adenovirus vectors with the 100K gene deleted and their potential for multiple gene therapy applications. J. of Virol. 75:5913-5920, 2001.
25. Huang M. J. and Hearing P., Adenovirus early region 4 encodes two gene products with redundant effects in lytic infection. J. Virol. 63:2605-2615, 1989.
26. Kay, M. A., Glorioso, J. C. and Naldini, L., Viral vectors for gene therapy: the art of turning infectious agents into vehicles of therapeutics. Nat. Med. 7:33-40, 2001.
27. Klessig, D. F., Brough D. E. and Cleghon V., Induction, stable integration, and controlled expression of a chimeric adenovirus gene whose product is toxic to the recipient human cell. Mol. Cell. Biol. 4:1354-1362, 1984.
28. Kochanek S., Clemens P. R., Mitani K., Chen H. H., Chan S., Caskey C. T., A new adenoviral vector: replacement of all viral coding sequences with 28 Kb of DNA independently expressing both full-length dystrophin and β-galactosidase. Proc. Natl. Acad. Sci. 93:5731-5736, 1996.
29. Lamartina S., Silvi L., Roscilli G., Casimiro D., Simon A. J., Davies M. E., Shiver J. W., Rinaudo C. D., Zampaglione I., Fattori E., Colloca S., Paz O. G., Laufer R., Bujard H., Cortese R., Ciliberto G., Toniatti C., Construction of an rtTA2s-M2/tTSkid-based transcription regulatory switch that displays no basal activity, good inducibility, and high responsiveness to doxycycline in mice and non-human primates. Mol. Therapy. 7:271-280, 2003.
30. Lechner F., Wong D. K., Dunbar P. R., Chapman R., Chung R.T., Dohrenwend P., Robbins G., Phillips R., Klenerman P., Walker B. D., Analysis of successful immune responses in persons infected with hepatitis C virus. J. Exp. Med. 191:1499-151246., 2000.
31. Lieber A, He C. Y., Kirillova I. and Kay M. A., Recombinant adeno-viruses with large deletions generated by Cre-mediated excision exhibit different biological properties compared with first-generation vectors in vitro and in vivo. J. Virol. 70: 8944-8960, 1996.
32. Lohmann, V., Korner F., Herian U. and Bartenschlager R., Biochemical properties of hepatitis C virus NS5B RNA-dependent RNA polymerase and identification of amino acid sequence motifs essential for enzymatic activity. J. Virol. 71:8416-8428, 1997.
33. Lusky M., Christ M., Rittner K., Dieterle A., Dreyer D., Mourot B., Schultz H., Stoeckel F., Pavirani A., Mehtali M., In vitro and in vivo biology of recombinant adenovirus vectors with E1, E1/E2, or E1/E4 deleted. J. of Virol. 72:2022-2032, 1998.
34. Lusky M., Grave L., Dieterle A., Christ M, Ziller C, Furstenberger P, Kintz J, Hadji D A, Pavirani A, Mehtali M., Regulation of adenovirus-mediated transgene expression by the viral E4 gene products: requirement for E4 ORF3. J. Virol. 73:8308-8319, 1999.
35. Marshall E., Gene therapy death prompts review of adenovirus vector. Science. 286: 2244-2245, 1999.
36. Mitani K., Graham F. L., Caskey C. T. and Kockanek S., Rescue, propagation, and partial purification of a helper virus-dependent adenovirus vector. Proc. Natl. Acad. Sci. 92:3854-3858, 1995.
37. Mittereder N., Yei S., Bachurski C., Cuppoletti J., Whitsett J. A., Tolstoshev P., Trapnell B. C., Evaluation of the efficacy and safety of in vitro, adenovirus-mediated transfer to human cystic fibrosis transmembrane conductance regulator cDNA. Hum. Gene Ther. 5:717-729, 1994.
38. Moorhead J. W., Clayton G. H., Smith R. L. and Schaack J., A replication-incompetent adenovirus vector with the preterminal protein gene deleted efficiently transduces mouse ears. J. Virol. 73:1046-1053, 1999.
39. Morsy M. A., Gu M., Motzel S., Zhao J., Lin J., Su Q., Allen H., Franlin L., Parks R. J., Graham F. L., Kochanek S., Bett A. J., Caskey C. T., An adenoviral vector deleted for all viral coding sequences results in enhanced safety and extended expression of a leptin transgene. Proc Natl Acad July 95:7866-7871, 1998.
40. Morsy M. A. and Caskey C. T., Expanded-capacity adenoviral vectors—the helper-dependent vectors. Mol Mem Today 5: 18-24, 1999.
41. Parks R. J., Chen L., Anton M., Sankar U., Rudnicki M. A., Graham F. L., A helper-dependent adenovirus vector system: removal of helper virus by Cre-mediated excision of the viral packaging signal. Proc Natl Acad Sci 93:13565-13570, 1996.
42. Rich D. P., Couture L. A., Cardoza L. M., Guiggio V. M., Armentano D., Espino P. C., Hehir K., Welsh M J., Smith A. E., Gregory R. J., Development and analysis of recombinant adenovirus for gene therapy of cystic fibrosis. Hum. Gen. Ther. 4:461-476, 1993.
43. Sandig V., Youil R., Bett A. J., Franlin L. L., Oshima M., Maione D., Wang F., Metzker M. L., Savino R., Caskey C. T., Optimization of the helper-dependent adenovirus system for production and potency in vivo. Proc. Natl. Acad. Sci. 97:1002-1007, 2000.
44. Schaack J., Guo X., Ho W. Y., Karlok M., Chen C., Ornelles D., Adenovirus type 5 precursor terminal protein-expressing 293 and HeLa cell lines. J. Virol. 69:4079-4085, 1995.
45. Schaack, J. Xiaoling, G. and Langer, S. J., Characterization of a replication-incompetent adenovirus type 5 mutant deleted for the preterminal protein gene. Proc. Natl. Acad. Sci. 93:14686-14691, 1996.
46. Shiver J. W., Fu T. M., Chen L., Casimiro D. R., Davies M. E., Evans R. K., Zhang Z. Q., Simon A. J., Trigona W. L., Dubey S. A., et al., Replication-incompetent adenoviral vaccine vector elicit effective anti-immunodeficiency-virus immunity. Nature 415:331-335, 2002.
47. Sullivan N J, Geisbert T W, Geisbert J B, Xu L, Yang Z Y, Roederer M, Koup R A, Jahrling P B, Nabel G J., Accelerated vaccination for Ebola virus haemorrhagic fever in non-human primates. Nature 424: 681-684, 2003.
48. Vos, J. M. H., Herpesviruses as genetic vectors. Viruses in human gene therapy. Carolina Acad. Press. And Chapman & Hall: Durham N.C., London, 109-140, 1995.
49. Ward S., Lauer G., Isba R., Walker B., Klenerman P., Cellular immune response against hepatitis C virus: the evidence base 2002. Clin. Exp. Immunol. 128:195-203, 2002.
50. Yang Y., Nunes F. A., Berencsi K., Furth E. E., Gonczol E., Wilson J. M., Cellular immunity to viral antigens limits E1-deleted adenoviruses for gene therapy. Proc Natl Acad Sci USA. 91:4407-4411, 1994.
51. Yang Y., Nunes F. A., Berencsi K., Gonczol E., Engelhardt J. F., Wilson J. M., Inactivation of E2a in recombinant adenovirus improves the prospect for gene therapy in cystic fibrosis. Nature Genet. 7:362-369, 1994.
52. Yoshida T., Okuda K., Xin K. Q., Tadokoro K., Fukushima J., Toda S., Hagiwara E., Hamajima K., Koshino T., Saito T., Activation of HIV-1 specific immune response to an HIV-1 vaccine constructed from a replication-defective adenovirus vector using various combination of immunization protocols. Clin. Exp. Immunol. 124:445-452, 2001.
53. Youil R., Toner T. J., Su Q., Casimiro D, Shiver J. W., Chen L., Bett A. J., Rogers B. M., Burden E. C., Tang A., Chen M., Emini E., Kaslow D. C., Aunins J. G. and Altaras N. E., Comparative Analysis of the Effects of Packaging Signal, Transgene Orientation, Promoters, Polyadenylation Signals, and E3 Region on Growth Properties of First-Generation Adenoviruses. Hum. Gene Ther. 14:1017-1034, 2003.
54. Zhou H. and Beaudet A. L., A new vector system with inducible E2a cell line for production of higher titer and safer adenoviral vectors. Virology 275:348-357, 2000.
55. Zucchelli S., Capone S., Fattori E., Folgori A., Di Marco A., Casimiro D., Simon A. J., Laufer R., La Monica N., Cortese R, Nicosia A. J. Virol., 74:11598-11607, 2000.
56. Cooper S., Erickson A. L., Adams E. J., Kansopon J., Weiner A. J., Chien D. Y., Houghton M., Parham P., Walker C. M. Immunity, 10:439-449, 1999.
57. Lechner F., Wong D. K., Dunbar P. R., Chapman R., Chung R.T., Dohrenwend P., Robbins G., Phillips R., Klenerman P., Walker B. D. J. Exp. Med., 191:1499-1512, 2000.
58. Ward S., Lauer G., Isba R., Walker B., Klenerman P. Clin. Exp. Immunol., 128:195-203, 2002.
59. Casimiro D. R., Chen L., Fu T. M., Evans R. K., Caulfield M. J., Davies M. E., Tang A., Chen M., Huang L., Harris V., et al. Gene. J. Virol., 77:6305-6313, 2003.
60. Mecsas J, Sugden B., Replication of plasmids derived from bovine papilloma virus type 1 and Epstein-Barr virus in cells in culture. Annu Rev Cell Biol.;3:87-108, 1987.
61. M. P. Calos, PNAS, 95:4084-4085, 1998.
62. Horlick et al., Prot. Exp. And Purific. 9:301-308, 1997.
63. Mayo K E, Palmiter R D, Glucocorticoid regulation of the mouse metallothionein I gene is selectively lost following amplification of the gene. J Biol Chem, 257: 3061-3067, 1982.
64. Palmiter R D; Regulation of metallothionein genes by heavy metals appears to be mediated by a zinc-sensitive inhibitor that interacts with a constitutively active transcription factor, MTF-1. Proc Natl Acad Sci USA, 91: 1219-1223, 1994.
65. Baim S B, Labow M A, Levine A J, Shenk T. A chimeric mammalian transactivator based on the lac repressor that is regulated by temperature and isopropyl beta-D-thiogalactopyranoside. Proc Natl Acad Sci USA, 88: 5072-5076, 1991.
66. Lee F et al. Functional analysis of the steroid hormone control region of mouse mammary tumor virus. Nucleic Acids Res, 12: 4191-4206, 1984.
67. Wang Y, O'Malley Jr B W, Tsai S Y, O'Malley B W, A regulatory system for use in gene transfer. Proc Natl Acad Sci USA, 91: 8180-8184, 1994.
68. Gossen M, Bujard H, Tight control of gene expression in mammalian cells by tetracycline-responsive promoters. Proc Natl Acad Sci USA, 89: 5547-5551, 1992.
69. Lopez-Labrador F. X., et al., Genetic variability of Hepatitis C virus non-structural protein 3 and virus specific CD8+ response in patients with chronic hepatitis C. J. Med. Virol., 72: 575-585, 2004.

1: An adenoviral amplicon comprising:
a. an EBV-derived origin of replication (Ori-P);
b. an Ad5 (ITR junction);
c. a first transcriptional unit consisting of nucleic acid sequences encoding Ad5-derived polymerase and preterminal protein;
d. a second transcription unit consisting of a nucleic acid sequence encoding Ad5 E4 ORF6 and DNA binding protein; and
e. a marker of selection; wherein the first and second transcriptional units are fused to a bi-directional tetracycline-dependent promoter.

2: The adenoviral amplicon of claim 1, wherein said amplicon comprises the nucleotide sequence of pE2

3: (canceled)

4: The adenoviral amplicon according to claim 1 further comprising an expression cassette encoding a transgene fused to a promoter.

5: An adenoviral producer cell which expresses:
a. an EBV-derived EBNA1 protein;
b. a Tet transcriptional silencer;
c. a Tet reverse transactivator;
d. an adenoviral amplicon consisting of: an EBV-derived Ori-P, an adenoviral ITR junction, and a first transcriptional unit consisting of nucleic acid sequences encoding Ad5-derived polymerase and preterminal protein in combination with a second transcription unit consisting of a nucleic acid sequence encoding Ad5 E4 ORF6 and DNA binding protein, wherein the first and second transcriptional units are fused to a bi-directional tetracycline-dependent promoter; and
e. a selection marker.

6: The producer cell according to claim 5, wherein the cell is a primate-derived cell line expressing a EBNA1 protein.

7: The producer cell line according to claim 6, wherein the cell is 293EBNA

8: The producer cell according to claim 5, wherein the Tet transcriptional silencer is tTS^{kid}.

9: The producer cell line according to claim 8, wherein the Tet reverse transactivator is rtTA2.

10: (canceled)

11: A method for producing replication defective adenovirus comprising a gene of interest, which comprises:
a. introducing a multiply-deleted adenoviral expression vector into a producer cell which expresses:
i. an EBV-derived EBNA protein;
ii. a Tet transcriptional silencer;
iii. a Tet reverse transactivator;
iv. an adenoviral amplicon consisting of: an EBV-derived ori-P, an adenoviral ITR junction, and a first transcriptional unit consisting of nucleic acid sequences encoding Ad5 E2-derived polymerase and preterminal protein in combination with a second transcription unit consisting of a nucleic acid sequences encoding Ad5 E4 ORF6 and DNA binding protein, wherein the first and second transcriptional units are fused to a bi-directional tetracycline-inducible promoter;
b. inducing expression of the E2 and E4ORF6 coding sequences; and
c. harvesting the replication defection adenoviruses which are produced.

12: The method according to claim 11, wherein the producer cell line is a human cell line expressing an adenovirus E1 protein, EBNA 1, and a transcription regulation system.

13: The method according to claim 12, wherein the producer cells are 293EBNA cells expressing tTs^{kids}, and rETA2.

14: The method according to claim 13, wherein the multi-deleted adenoviral vector lacks adenoviral E1, E2, E3 and E4 genes.

15: The method according to claim 14, wherein the multi-deleted adenoviral vector consists of a human Ad5 backbone.

16: The method according to claim 11, wherein expression of the E2 and E4ORF6 coding sequences is induced by contacting the producer cells with doxycycline.

17: A method for producing replication defective adenovirus particles which comprises introducing an adenoviral amplicon according to claim 4 into mammalian cells expressing EBNA1, a Tet transcriptional silencer and a Tet reverse transactivator; inducing expression of the E2 and E4ORF6 coding sequences; and harvesting the replication defective adenoviruses which are produced.

18: The method according to claim 17, wherein the producer cell line is 293EBNA cells expressing tTS^{kid} and rtTA2.

19: The method according to claim 17, wherein expression of the E2 and E4ORF6 coding sequences is induced by contacting the packaging cells with doxycycline.

20: Recombinant replication defective adenovirus particles harvested and purified by the method according to claim 17.
